SEQUENCE LISTING (17 sequences)

SEQ ID NO: 1 - DNA, 1551 bp, Mus musculus
cgccgagcgccgccgccgccgccgccgccgccgctccgctgcccgcgccgcccgcggctcccgatggagactgacgcgccccagcccggcctcgcctccccggactcgccgcacgacccctgcaagatgttcatcggaggactcagttggcagaccacgcaggaagggctgcgcgaatacttcggccagttcggggaggtgaaagagtgtctggtgatgcgggaccccctgaccaaaagatccaggggtttcggcttcgtcactttcatggaccaggcgggggtggataaagtgctggcgcaatcgcggcacgagctcgactccaaaacaattgaccccaaggtggcctttcctcgaagagcacagcctaagatggtcactcggacgaagaagatcttcgtgggggggctgtctgtgaacaccacggtggaagatgtgaaacactatttcgagcagttcggaaaggtggatgatgccatgctgatgttcgacaaaaccaccaacaggcacagagggtttggatttgtcacgtttgagagcgaggacatcgtagagaaagtttgtgagatccacttccatgaaatcaacaacaaaatggtggaatgcaagaaagcccagccaaaggaggtgatgtccccgacaggctcagcccggggcaggtctcgggtcatgccctacggaatggatgccttcatgctgggtattgggatgctgggttacccagggttccaagccacgacctacgccagccggagttacacaggccttgcccctggttacacctaccagttccccgaattccgtgtagagcggagccctctcccgagcgccccagtcctccccgagctcacagctatccctctcacggcttatgggcccatggcggcggcagcggcggcggcagctgtagttcgagggacaggctctcacccctggacgatggctccccctccaggttccactcccagccgcacagggggcttcctagggaccacaagccccggccccatggctgagctctacggggcagccaaccaggactccggggtcagcagttacatcagcgccgccagccccgcccccagcactggtttcggccacagtcttgggggtcccttgattgccacagccttcaccaatgggtaccactgaaacagggaggaggtagcaggagcgccccagcctgcagctgactgaggaccagactgagccagcaaggggattgggacacctccgccgcagcagcccagccccttggctgccacttggaccgctactgcctgtccctcaacccctgggcccagccccctcatgtctggctcccctactaacctcctgttcagaccttgtctcttctctcgctcccacctgcctctctccctggtcgcttttatttatttttggattagccagttgccctacccccacaccagatctgccctctcctccggtctgccccatccctccctgctgccccctttagggcaccccccccccagaaaggcatagctggagggcgggcagagggggcctgctgcagactgaggcccc

SEQ ID NO: 2 - DNA, 6412 bp, Mus musculus
ggagcttcgcagccgccgggcggggggaggaggcgggggaggagggacggagatctggggctcggagccggccgccgctgcgctccgcttcgctgtggggctcggtgtgtcgggggtgggggggcgggggggctccgctatggaggcaaatgggagcccaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccacaacgaaacgctccagaggcttcggtttcgtcaccttcgcagacccagcaagtgtagataaagtattaggtcagccccaccatgagttagattccaagacgattgacccaaaagttgcatttcctcgtcgagcgcaacctaagatggtcacaagaacaaagaaaatcttcgtaggaggattgtctgcaaacacagtagtggaagatgtaaagcagtatttcgagcagtttggcaaggtagaggatgcgatgctgatgttcgacaaaaccaccaacaggcacagagggtttggctttgtcacctttgagaatgaagacgttgtggagaaagtctgtgagattcatttccatgaaatcaataataaaatggtagaatgtaagaaagctcagccgaaagaagtcatgttcccacctgggacaagaggccgggcccgggggctgccatacaccatggatgcgttcatgcttggcatggggatgctgggctaccccaactttgtggcaacctatggcagaggctaccccggatttgctcctagctatggctaccagttcccaggcttcccggcggcagcttatggaccagtggcagcggcagctgtggcagcggctcgaggatcagtcctgaatagctacagtgctcaaccgaattttggcgcgcccgcttccccggcaggctccaacccggcgcggcccggaggcttcccgggggccaacagcccaggacctgtcgccgatctctacggccctgccagccaggactccggagtggggaattacataagcgcggccagcccacagccgggctccggcttcggccacggcatagctggacctttgattgcaacggcctttacaaatggataccactgagcaggcgcttccattgccgtctcactatgagagcatacctggatgtccaggcaagactgggcgaagtttctgagtggccctttgtttaggtgacgtcctcagacctggacccccaccagcctcactcctcatcccaaccagaggtggcacacttggattgagggttgacacatctcatctcacccatcggctacctgctgtaatataagacaacagcttttaaacgtgtatataatccatgattttggtttggttctgtttgttttccttggtggtccccctctccctcttcccttctccttttaaatctccctcaatcacatttggtagtgatttttgacttagtctggtagtcacccagcttaatatctagttaaagctaaccatagtatacttgttatatattaaggagttttttttctttttttttgttttcttttttcctttaaagagaatttttgttttgttttgattctgttctcgcttttaaaggatgctgagatggtgatgttactctccatttttggtaccagttctgagactgtaagacttttcatctgggactttagcacacactgaatcgaagttgtgatacgcggagcgggaggtgggcatagactctattttgtgttgtagaagtgacatacagttggctgcttaacagactctctagccgttcatttttgtgacgtctctttgttaacctaagtatatctattttcggcaataaggtaaggacggccgtgttttgagggtcttcctttcctatgagtgctttttcttttcttctgttcaaagaggtcatattacaaggttttttgaaattgtgaattctaaaaaacaaaaaacaaacaaaaaaatgttgtaaatataattccattaactacatagaaactattaaggaagagagaatcaaaaaatatttttgtgagggagttgatcctgggcagttaggtaccctggaaggaggggagggaacctcgatgaagccagtacaacaacagaagagcccccccagctgccagggcaccccagacccccactcttcactgttggtgtgatagacctaagaaccacattagcaccctgccagcctaccagcctctctgtgcacagcttctaatgttatgatctagctagatttttatttaaaatatgaaaaactgcttttcccaagacgttttttaaaaggaaaaaaagctacaattttaatatttgacatatttaaagtttcaaagcacacttgttttggcttaggggaggggcagggtggggtcattctttttcagtcttaatttttaaatatctgatcattttatattgtccgatcatttcagcacctccaaagggtcctaggatattctgcctctctcttcaccctgtccccagacgggggaacccgtgggtcaggcgtggataagcctgcgagaatacaccctttctccattcactttctatttacaaattaggaaagcaacctttcagtttatatatatattttttttcctttttgatacctcagtgctgcaagtatcatcagagaggctatggaagagttttgtgttttgttttttgttttgtttttttttttaatttattgtagatgtaaacagaattttaaaaataaaatataaacatcactgcactgtgactggtggggaaaactgacagcttcctgtttgtacacatttgacattggctgttataatatatggtcctcagttggggaagacacttagaatgaaggatgttttttaatttaacttttttttttttttaaagaaaaatatcagtaataggtcggcaacagcagccgtaggaagtacaacttagggtagcattaaagcatactgtagtgtggatatattttttttcttttttaaaatgtgatattgacattttattaatattttttaaattgttacgtttataaatttggtacttaaggcacagccagtgtgaggcactgaacgcgacatttattacaatgagctgctgcactcctacttttataaattttactaacaagtagactaatgtagacattcacagacgggatagcgcagaagcatcttcacacgacaagtctccaaaaaaaaaaagctaattcagagagggcgggaggaagcctgttcccgttctaattaagtgcaaccttgttttatcttattgttttattttttaagaaaggaaatcatgttctttttttatatctctatatataagatacagagttgtttggtaaggtttggggtgttttttgttttgttttgttttaagtttccaaacaaccaaactaagaaaaatgctggatgcttttagaatacaaaacttactcaagtcataaaataacaaaatagaacttttactttaagaaacaacaacaaagagagagagagactcgattcccagcaagtcagtgtctcacaagggcactggctaattactcttctgtccgcgttgctgatgtcctgtccccagcccctgtccatctcccccacactcccatcacctcatagaacgctctttgttgatcattgtctgttaataatgtataaaatggctatcttgtaagtgtgctgtcttggtactagtgtagtgactttttttctcctcttctagtacatattgataggtataatgtaattaaggttttaaaaaaaatagacatagttattcagattaggaccagtaaggatagaactttctcttatttatgaaaagaaaaaaaatgctaataatttgggggcagttttttcctttttttttttttccaagttcaaatttacttttatttttgctgatttgatgtggtttcaactaacccaaggtctcacaatgttcaaatggcggctgactctacagcattctgtaggtccctgcccccctagctgaaggggtgtgccatactaccttaaatgcaaacactagatatgcaaaactggatttttttaatttattttttaaaagagggaggtgtggtatattaaaatgattttactaagagaaaaaaaatatttttttaaggatgctcagaagaaattgataatctgtgtgaatatgttttagatgtttatatacattttgaagagacccagtagcccatagcacaaatcttgtgaacatctgatatgttttcaagtggctacctaggataaggttcatcattagtacccccacccccaccccaccgccatctagaagtccatcttaaacaattttttgtaaattctttcagcactggtgtccatctttgtctttgtttcagttaagctcaatagcgaatgtgggaccccctcctctgaccttccctgggggagaaaccctcttggctaatggctcttccctggcattatcaataaccacccggggactctctggctttcagatccatctgcctgagaccccaaggtcctctcctccctagaggggaggtggagcagcagttgactcctggttccctccctatttcagcctggatgtggggctggtggagatgcctccaccccaggagcctctgcatatgtggttcgtggccttcttctcacccattgggaaaaccaaacagcctcacactctgtccccatcgctcattggcttaactcaagtgagaccccaactgggcctttgcgttttgtttttgtttttttttaaatccttcatgaccattctcttaatttgaactcgtagcttgggctttaaggtagcatggctcgattgctgtcgacttaatgttccactgcacagcaattcacggccagtgaatgttacacacatcttgctagactagtataaaaatcattgggtaattgttggttctaatgacctgaaaggtgttcagtttgtgggttttttttggggggggctgcttggggggttgtttttccttctttgtttttttttaatttgagaatttagggggataatttttgggggggggttccaaataaaaaaatagaaagctattttgatctttagtgcaaactagggacgtagcctccatcaccacaaccacaccgcttcgctctgccatccacccaccgcagcagcatcttcaagaataaagcaatatagtttactacatttttttttaaaattgaaggtcagccatgctttctgtattatattgcatatgaaattgtttacaaaagaaacactaactcatccttctctttatcagttgaaagtgacacacgggacagagggaaattggagggcaggtgagggaggggagagggtttttttgtattttggtttgtttggttttggtttcttttcttgaatgtgctttagtagccagatcctccaaggaaggcaaaagccagccagccagagagagagtagcgaagcgggcaggctgcaggtaagcacactgaacaagagacacactggcaagctcccccacctcagcgagagcagaaagcgaacagctcagcccagtcctgggcacagacactacacaaggcaatgctggaattgaaacaatactttaataccgtttaagtttgtttcctttttttttttcctcttttttttttctttcaccaagaaaagaaaaaagtaaactaaaatacaaaatacaaaaacaaacaaacaaacaacaacccatataaataaaacccacccctctctgggaaacaaccataattcaaacatggctatttagtaatcagaaggtattgtctcagacaggatttcatttccgggaggcagggcgtgaggggggaggggagtgggactgagagacagttccagagcctccagggaaggcttctactgctaactgctgtattctgtatatactgtgccaccctgtgtggagtctgtgagtgtgctcttgagtagcgtgggctagccaatctgccattcatggtgttataaactcggaattccatatgtaataggatgcaagtctaagcgtttcatgtggacataaatgtatctaaataaaacttcccctagcactgtggctgacctcacccttacttttatactttagtatgaaactgatgagaactttggtagtgagtattttttttatatatatacatatatatgtatctatatatatatatatatctcaagcatctttcaggtctttgtgtgtggctttcttaaagccctgttgtaaaaaaaaaaaaaattactaagtggatggcagtctctcacatcacagatgtggaaagtataattttatatttgtattttcaaataaataagtttgtgaaaggt

SEQ ID NO: 3 - DNA, 2950 bp, Homo sapiens
gcgccgagcgccgccgccgccgccgccgccgccgctccgctgcccgcgccgcccgcggctcccgatggagactgacgcgccccagcccggcctcgcctccccggactcgccgcacgacccctgcaagatgttcatcgggggactcagttggcagactacgcaggaagggctgcgcgaatacttcggccagttcggggaggtgaaggagtgtctggtgatgcgggaccccctgaccaagagatccaggggtttcggcttcgtcactttcatggaccaggcgggggtggataaagtgctggcgcaatcgcggcacgagctcgactccaaaacaattgaccctaaggtggccttccctcggcgagcacagcccaagatggtgactcgaacgaagaagatctttgtgggggggctgtcggtgaacaccacggtggaggacgtgaagcaatattttgagcagtttgggaaggtggacgacgccatgctgatgtttgacaaaaccaccaaccggcaccgagggttcgggtttgtcacgtttgagagtgaggacatcgtggagaaagtgtgtgaaattcattttcatgaaatcaacaacaaaatggtggaatgtaagaaagctcagccaaaggaggtgatgtcgccaacgggctcagcccgggggaggtctcgagtcatgccctacggaatggacgccttcatgctgggcatcggcatgctgggttacccaggtttccaagccacaacctacgccagccggagttatacaggcctcgcccctggctacacctaccagttccccgaattccgtgtagagcggacccctctcccgagcgccccagtcctccccgagcttacagccattcctctcactgcctacggaccaatggcggcggcagcggcggcagcggctgtggttcgagggacaggctctcacccctggacgatggctccccctccaggttcgactcccagccgcacagggggcttcctggggaccaccagccccggccccatggccgagctctacggggcggccaaccaggactcgggggtcagcagttacatcagcgccgccagccctgcccccagcaccggcttcggccacagtcttgggggccctttgattgccacagccttcaccaatgggtaccactgaagcaggggacggtggcaggagcgccccagcctgcagctgactgaggaccacgagtgagccagcgagggggcgggagacctcagccgcagccgccgccccctcccctgcagcgactcggacccgctactgcctgcccccaactccccgggcccggcccctgccctgctgcccccaacagcgtctggctcccctactaacgtccccctcttcgcccttgcccccatccccacccgcccctctcccggccctgcttttatttattttggattagccggttgcccaccccagccctctggtccatccctccctccgtgccgcgcccccctaggaccgccccctccccaaaaggcttttggatttgtgcatagctggagtgaaggcggagggagcctgctacaggccgcagcccaacccctgttttttattcagatttcccctcctttaccctttccctttttttttttttttttttttttaaagaaaccttttttaaactatttctaggtttgtgaatgtgaagccccaggccgcagggggcaaggggccaggtgccccccaccagctgagaacaaagtgtctatctgggtgtgggcccctggccgcctccctccagccctggagaggagggcagggctgcggggaggccaggccgagcccctggaaccatcccgtcctgtatcatatgtaaatactgtgaggtgatgtgcccacccctctctaagacccctcgggggtgaggggctcccccctcccctgtttctgtcccctcagacaccgttactgtaagcttgcaggcctcagctgtggccacggcaggcccgctctctcaggccctcagggtcaaggccttggttggacctgcccactccaaaaacccagtgtgggggcaaagggcgtgggaagagcagggcttgcccagcgacactgctggacaggaattaactctccaaaggtctcccctgctccctacctaggttggggcttcatggtttctgctcagtctgtcccccttccccctcgacccccgcaatgagtgggcaccaggggacgctctggcgagggcagaccccaggggaaagcaaagggcgtctcagggaacccccacattctctcactgaagtttcccaccaggatgaccccacagccagagtcccttggcagcccctcaccccagaccccccttctaaggaaaaagagaggttcagagcgttggaccttcatgaacagtggcctggctggcgtggcagggccaaggcccacccactgccatcccccttctgtgtgtccctctcccccagttatgaggcccagggcctgagctctcttccccagcattgcccccacccggaaaccccacctttggagagttaattgtctgtgtgaggtgcttaacctatcagccctgagaacacaaagcaataatctttgttactgagatgcgcggctgttcgtgttttgggttttttttttttttaatgtttcctaataaaagagaagctgcattttattggtttttatttttaattttctacacgtttgagctgagtcctgagacacttagcttccctctccctcattcccggacccttccaccccactgggcccaaccatgggctcaggaccctggaattccgttttctgatttgctttgggattttttttttttttaagatgttacatggtgtttcgaagccagcaagttaccatcctccggtgtctcttctctccacatctgtaacttctttttccaggttttattttcagttttaaattcctaataaattatttgaaaacgtt

SEQ ID NO: 4 - DNA, 1581 bp, Homo sapiens
ctcgccgctgccccggctccgccgctcgcagagagattcggaggagcccgggcgggggggaggaggagggggaggagggagcggagatctcggggctcggagccggccgccgctccgctccgatcgctgtggggcttggttttttgggggtgggggggcgggggggctcagatatggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttat

SEQ ID NO: 5 - DNA, 2431 bp, Xenopus laevis
gtggctacaccgccccttccaatcctcttgtccgcgggagctgcctccgatctcaggagtcgccagagaccgaatggagacagaagcgccccagcccggactgtcgcccccggactccgcgcacgatccctgcaaaatgttcattgggggactgagctggcagacaacacaagagggactgcgggagtacttcagtcattttggggatgtaaaagagtgtcttgtcatgagagatccattgaccaaaagatcaaggggtttcggatttgtaactttcatggaccaagcgggagtggacaaagttttggctcaatcacgacatgagctggactcaaagacgattgatccaaaagtagcatttcctcgtagagctcaacccaagatggtaacacggacaaagaagatttttgtagggggcttgtcagtgaatacaacagttgaagatgtgaaacagtattttgagcaatttgggaaggtggatgatgccatgttaatgtttgataaaacaacaaacaggcacagaggattcgggtttgtgacttttgaaggagaggatattgtggagaaaatttgcgatattcacttccatgagatcaacaataagatggttgagtgtaagaaggcccaacccaaggaggtgatgtcaccaacaggatctgtgagaggccgatctcgggtcatgccctatggaatggatgctttcatgctgggaataggcatgctaggatacccgggatttcaggcagctacatatgccagtcgcagctacacaggcattgctcctgggtatacttatcagttcccagagtttcgtgtagagagaacccctctcccgggcgccccagtcctcccagaactcacagctatccccctgacggcatatggtcctgtagcggcagcagctgcagcagtggtgagaggctccactccaacccgcacaggggggtttcttggaacaagcagcccaggaccaatggcagaactttatggagcagccaatcaagaatcggcagttagcagctatatcagtgcagcaagcccagcacccagcacaggctttggacacagccttgggggtcctctgattgccactgctttcaccaacggctaccactgaaacagcaagtcactgcagaggcttgagtctggaagcatctcttgcacgattctgttcctcccctctctgaatatcacatggctgtacctcaccttgtgttcctgcacagtaactggtgcctcctgcacaaccttcacctaggaatttcttatttatttctatttttgttttggggtttttttttcattctccttttattagatttgtgttttgtccaaattagaaatcagggagacatttccataatcttcatggttattctgttctggtaagatgcagagaatcctcggtacgaaacgattctgcagagagagaggggagattgtatttcgactgtctggaaatgtgttttttttcttcttttttttttatatttctatgtaaatttcggctcttaaaaacatgtaaaactgaaatttggagacaacagtgcacaagaacaagacaaatgtatattttgctaaatgaaaaagaactatttgtagaacagaaattattttaattttctcaaagctggtttgaagtttttttttttctgtgtaaaatatgtaaatattgcagactgattgttctgccaccggaaaggtctctttggcccagattatcttttttgcctacttgggcctgtgttttagcaatattttcctaaagcctgaatggattgtcgggtttttttatacatcttttattatgatggatgtcagtccacaggaatatgatgagttactctgatgcaggggaatgcagatgccatgctgcaaaacttcttactctttagcctctgataattaactctttaaggctcacttgtcctttgctcatggatataatgagagggctatatggagggctataaggtgggactaacctcagagaaccagaaaagctacagtaaagtgctgttacacactgacatacttcctcagaaccctcagaaaagtaagtgtatgcccagtcccgatttatgaactgtctgccctttgatctgctctgcactctttatagttttgggtcctataaattaaatgcagatctaccttttctttatgtgcagtgtgtaagtccacattttttgttatgaggaatcttcttaacaataaaatcatgtggaattagcccccaaccaaaccccccccacaaactgtctctcacgtgagcaaaggaaggtgctacactataaagagtttcttatcactgggccacacacattactgcaatgtacaatgtactgcaacgtcaaagcaataatctttgttactgcaactggacttttttaaaagatagttcaaaataaactatgaattttattcattt

SEQ ID NO: 6 - DNA, 1774 bp, Xenopus laevis
tttttgtattactgtcattgctctctatttgtctaagtgtgttgagaaggctatggaggcagatgggagccaggccacctctgggagtccgaacgattctcagcacgaccccggtaaaatgttcatcggcggcctgagctggcagacctccccagatagcctcagagactattttaacaaatttggagagatcagagaatgcatggtgatgagggaccccaccacaaagcgatccagaggcttcggtttcgttacgtttgcagatcctgcaagtgtagataaagtattagctcaaccccatcatgaattagattccaagacgattgacccaaaagttgcatttcccagaagagcacagcccaagatggtcacaagaacaaagaaaatttttgtaggtggattatcagcgaacacggtagtagaagatgtaaagcaatattttgaacagtatggaaaggtagaagatgccatgttgatgtttgacaaaacaacaaacagacatagaggttttggctttgtaacctttgaaatagaagatgttgtggagaaagtatgtgaaattcatttccatgaaataaacaacaaaatggttgaatgtaagaaagcacagcctaaggaagtcatgtttcctcctggtacgagaggacgagcaaggggtcttccttacaccatggatgcgttcatgctagggatgggaatgctgggttacccaaattttgttgcaacgtatggccgaggatatcccggatttgccccaagctacagctatcagtttccaggttttcctgcagcaacaacttatggaccagtaacggcagcagctgtggcagcagctagaggatcaggaatcccagattatggtttctataccacgcccggtgaccaacgggctacgttatcgtttgcggactacggttccatggggcctcgaacagctcagatgcttcgtagtgagcatgctgcttcagcaagtaattcccccctccatcaccttcccagcccggatcagtttaagagcccagtcttgaatagctacagtgctcaaccgaattatggcacacctgcttccccgacaggcaccaacccgggtcgtccaggaggattcccaggggcaaatagcccaggcccagtcgctgacctctatggaactgccagccaggactccggtgtgggtaactacattagtgcggcaagtccacaaccaggctctggctttagccacggaatggctggccctttgattgcaacggcttttacaaatggataccattgaaacggttcagatggttggttgccatctcactttgagactttgtctggatgcccaagcaagactgggagaagtttttgaatgttcccttgttagaataatgtcatcggctcttaaatacattgtcctcacaagtacaagacatatatgcagacgtaatatcggaggtggcattctgcgaactacagtacatctcgtcatctcatgaacctgctgtgttaaataatccacagctttttcttatgggaaaagcaagtgcaaaagtcatttattgttttcattattgcttgtgttttatttttctcctttgtattcaggcatttggggttaaggatctacagcttggggtttccattttagttgatttcatatgtttgaagttaaccatagtatgcatcattattaatggcttacttttttttttaacatggttactaatgcgcgttgcaaatacttaagattttatagattttaacagaatttgcaagcattgggatgtcgcac

SEQ ID NO: 7 - DNA, 4091 bp, Drosophila melanogaster
tagttaaaaaaaattccttgcccgaagcggaaggattgtcgaatttcgtggatttttgcgaatatttcccgtgtaattttcgtcacgcgtcgtttatttgcaccagcgagtgtgtgtgagtgtgtgtataacgtactggtgtgtgtgcgtaaaatctgaaaccttgccatctaatattggtggtattaaaatgagtattacctaagaaaaacaaaaataattcagtgcggttataaaatacataatatttaacaaaatagttgcaaaaagtcaacagaaaagtgcaaaaagaaaaaaatctcaagaaaaacaaagtatttggtgcgacctaaagtgattttctcagcgtgtgtctttgggataagcgaaaaaccagcaaaatttactgctttggaaactccgctctcaacataagcaccaacaacaacaccatcaacccccccggcggcagcagcacccacctccagattcagcaccaccaggctcagcaccacccccaaaacagtgcgagaatgctgttcgagaatccggcggttgcggccaagttgccctttccgtacaacgtgcccccgcccctgcaggcggcggcggcagcagcggcggctgtcccaaatctaaggtttcagacacccattaaagcgtttgcctgcctgacagcagccacaaggtcagtatcggagatgaacgccacttctctatatgccggcaatcccatggagaacgcggccgcagcagccgccgccgcagcagcgggcttgattgatccgcaccacaatcgcgatctccaccaggcgctggtggcctcgatagccaataatagtgtggccgccatcggcggtggcctgaccacagcggcagtcttgaagagtgccgctcagcagtcccagcaggcggtgcaacagaaccagaacgcggtagtggtgacaccgggactggaacagcccaaacaggaaccagcccaacaggcggcactggcgctgctcaaggagaatgtaaacgcctccgccggagcgggacagaacaacggtcaggcggccatgggtggcagcaacaagagcggctcctctggcagatccacgcccagcctgagcggaggcagtggctccgatccggcacccggcaaactctttgtcggcggtctcagctggcagacgtcgtctgacaagctcaaggaatacttcaacatgttcggcaccgtcaccgatgtgctcatcatgaaggatcccgtcacccagcgcagtcgcggctttggtttcatcacattccaagaaccctgcaccgtggagaaggttctcaaggtgcccattcacacactcgatggcaagaagatcgatcccaagcatgccaccccaaagaatcgacctcgtcaggccaacaagaccaagaagatcttcgtgggcggcgtctcacaggatacttcggccgaggaggttaaggcttacttcagccagttcggacccgtcgaggagacggttatgttgatggaccagcagacaaagcgccaccgtggcttcggatttgtcacctttgagaatgaggatgtggtcgatcgcgtatgtgagattcacttccacaccatcaagaacaagaaggtcgagtgcaagaaggcacagcccaaggaagcagtcacaccggctgctcagcttctccagaagcgcattatgttgggcaccctcggcgtccagctgcccacagctcctggccagctgattggagcccgtggtgccggcgtggccaccatgaacccactggccatgcttcaaaatcccacacagctactgcaatccccggcagcagccgctgccgcccagcaggccgccctcatatcacagaacccatttcaagtacaaaacgccgctgcggcagcctcgattgccaatcaggctggcttcggcaagctgttgaccacatatccgcagactgcgctgcatagcgtcagatatgcaccctactcgatccccgccagcgccgccactgccaacgccgccttgatgcaggctcatcaggcgcaaagcgtggccgccgctgcccatcatcaccagcagcagcaacagcagcagcatcatcaccagcagcagacccacaatgcccatgtggccgccgcccagcagcagcagcagagccaccacaacgccgtctcgaatcccgcttcgcaggcgcactcggcagcagcggctgccgccctggcggccaatgccgcgaatggggcaggtgccgctggagcccacagcctggccgctgccgctcagcaagctggcttaatggccggcaatcccctgaatgccgctgctgccgccgccgccgctgctgccaatccggcggctgcctactccaactatgcgctggccaacgtggacatgtccagcttccagggcgtcgattggagcaccatgtacggcatgggcatgtacgtctaagacgcgagttttgtttggtaatttgaatgaaacactctcaccatgcatacaccattcacaaccaccccccatacacccacaaccactaccacatgtgaacagcagccccccacatccattaatattcatagaaccacacacaaacactcatacgaagttggttttcctttgagtttggctaagttagtagtccgatacacggaaggcaaattaagtattatatttaaggcgccctcgagcgagttcgataaatactaactaatatatatatatataacaaatcataagcaatataagcgagaaaaaagaacaaaaacggaagcaaagcaaagcgaaggaaaaacaaaatttaaaaccaaaaagcaaaaattacggagcgttagagaagcaaaagaagaaaggaaaacacattaaaaaaccaccgattgtgattgcgtttaaatgtagccgttaatatataatttttagtgcataaaagaaaaccattaaatctaaattaaaataaacccaaataaaaactaaacgtatatataaatatacatacatatttataaaatgcaagagcaaggaatgaatcatataagttaactcacgctcgtataaccaagaatggcgctcactaattaaaaagttcgaaaaattgttcttttttttggagccctaaactcagctgaaatgaatgagaaactcctgcaagggcagtattaattaattgaatgaaacagcgaagaaaaacgttttgctcaagacaaagaactattcattatttttttaatcagcgatgaaaccctatattagactattattttagatgtttagttgagttcgaatttaattcacccagcatagaatacaacaacagagttaaccaatcactaacgaacatcaccaactcaccaacacgaaaactccaatatcctcctaccttctatctacccaaatctgttattgtacttaattatataacaattacaaataccacaacgaacaaaccaaaataacgttgtcttttatgtgtgcgactcttttttgatttagttctataactagtttattctgcactcaatctcaaacatttgtagcaagtaactaaataaccatttgttgaacattttggcatttgcatattcttattatactttcgagtgacgacaagttctcaaggctaccggcttcatttatagatcctaagatattcaaccgagaacgagtccttagggcactactaatattttagatccttcgacctatttcccaatcaccaagtctttcatttgcataaccatccaaacagctcatgaaccaggtaacataactttactgtagcttattgttttaaatggcacaatcttagttattaatattattttgtaattacccttatctgtagcatttgcatttgattttgcattcggcaaatacttcacccattgaaaccaagaaaattatcgaattaagaagaaacccggggcagatgctcctggatagttatgtatttgtctcatttgaatcgcttgcatgcttgaagtttaattttagttactagttatcatttggaaagtaatattttgcgtatgtaaatatctagtttatatacattacacacccacacaaaccacacattatatggaaaacaatgaaacatttgaagtaatttttgtgtttaataaaaac

SEQ ID NO: 8 - protein, 16 aa, Artificial Sequence
  Musashi 1 pepetide
VSSYISAASPAPSTGF

SEQ ID NO: 9 - protein, 16 aa, Artificial Sequence
  Musashi 1 peptide
VSSYLSAASPAPSTGF

SEQ ID NO: 10 - protein, 16 aa, Artificial Sequence
  Musashi 2 peptide
VGNYISAASPQPGSGF

SEQ ID NO: 11 - protein, 362 aa, Homo sapiens
METDAPQPGLASPDSPHDPCKMFIGGLSWQTTQEGLREYFGQFGEVKECLVMRDPLTKRSRGFGFVTFMDQAGVDKVLAQSRHELDSKTIDPKVAFPRRAQPKMVTRTKKIFVGGLSVNTTVEDVKQYFEQFGKVDDAMLMFDKTTNRHRGFGFVTFESEDIVEKVCEIHFHEINNKMVECKKAQPKEVMSPTGSARGRSRVMPYGMDAFMLGIGMLGYPGFQATTYASRSYTGLAPGYTYQFPEFRVERTPLPSAPVLPELTAIPLTAYGPMAAAAAAAAVVRGTGSHPWTMAPPPGSTPSRTGGFLGTTSPGPMAELYGAANQDSGVSSYISAASPAPSTGFGHSLGGPLIATAFTNGYH

SEQ ID NO: 12 - protein, 328 aa, Homo sapiens
MEANGSQGTSGSANDSQHDPGKMFIGGLSWQTSPDSLRDYFSKFGEIRECMVMRDPTTKRSRGFGFVTFADPASVDKVLGQPHHELDSKTIDPKVAFPRRAQPKMVTRTKKIFVGGLSANTVVEDVKQYFEQFGKVEDAMLMFDKTTNRHRGFGFVTFENEDVVEKVCEIHFHEINNKMVECKKAQPKEVMFPPGTRGRARGLPYTMDAFMLGMGMLGYPNFVATYGRGYPGFAPSYGYQFPGFPAAAYGPVAAAAVAAARGSGSNPARPGGFPGANSPGPVADLYGPASQDSGVGNYISAASPQPGSGFGHGIAGPLIATAFTNGYH

SEQ ID NO: 13 - protein, 362 aa, Mus musculus
METDAPQPGLASPDSPHDPCKMFIGGLSWQTTQEGLREYFGQFGEVKECLVMRDPLTKRSRGFGFVTFMDQAGVDKVLAQSRHELDSKTIDPKVAFPRRAQPKMVTRTKKIFVGGLSVNTTVEDVKHYFEQFGKVDDAMLMFDKTTNRHRGFGFVTFESEDIVEKVCEIHFHEINNKMVECKKAQPKEVMSPTGSARGRSRVMPYGMDAFMLGIGMLGYPGFQATTYASRSYTGLAPGYTYQFPEFRVERSPLPSAPVLPELTAIPLTAYGPMAAAAAAAAVVRGTGSHPWTMAPPPGSTPSRTGGFLGTTSPGPMAELYGAANQDSGVSSYISAASPAPSTGFGHSLGGPLIATAFTNGYH

SEQ ID NO: 14 - protein, 346 aa, Mus musculus
MEANGSPGTSGSANDSQHDPGKMFIGGLSWQTSPDSLRDYFSKFGEIRECMVMRDPTTKRSRGFGFVTFADPASVDKVLGQPHHELDSKTIDPKVAFPRRAQPKMVTRTKKIFVGGLSANTVVEDVKQYFEQFGKVEDAMLMFDKTTNRHRGFGFVTFENEDVVEKVCEIHFHEINNKMVECKKAQPKEVMFPPGTRGRARGLPYTMDAFMLGMGMLGYPNFVATYGRGYPGFAPSYGYQFPGFPAAAYGPVAAAAVAAARGSVLNSYSAQPNFGAPASPAGSNPARPGGFPGANSPGPVADLYGPASQDSGVGNYISAASPQPGSGFGHGIAGPLIATAFTNGYH

SEQ ID NO: 15 - protein, 347 aa, Xenopus laevis
METEAPQPGLSPPDSAHDPCKMFIGGLSWQTTQEGLREYFSHFGDVKECLVMRDPLTKRSRGFGFVTFMDQAGVDKVLAQSRHELDSKTIDPKVAFPRRAQPKMVTRTKKIFVGGLSVNTTVEDVKQYFEQFGKVDDAMLMFDKTTNRHRGFGFVTFEGEDIVEKICDIHFHEINNKMVECKKAQPKEVMSPTGSVRGRSRVMPYGMDAFMLGIGMLGYPGFQAATYASRSYTGIAPGYTYQFPEFRVERTPLPGAPVLPELTAIPLTAYGPVAAAAAAVVRGSTPTRTGGFLGTSSPGPMAELYGAANQESAVSSYISAASPAPSTGFGHSLGGPLIATAFTNGYH

SEQ ID NO: 16 - protein, 406 aa, Xenopus laevis
MEADGSQATSGSPNDSQHDPGKMFIGGLSWQTSPDSLRDYFNKFGEIRECMVMRDPTTKRSRGFGFVTFADPASVDKVLAQPHHELDSKTIDPKVAFPRRAQPKMVTRTKKIFVGGLSANTVVEDVKQYFEQYGKVEDAMLMFDKTTNRHRGFGFVTFEIEDVVEKVCEIHFHEINNKMVECKKAQPKEVMFPPGTRGRARGLPYTMDAFMLGMGMLGYPNFVATYGRGYPGFAPSYSYQFPGFPAATTYGPVTAAAVAAARGSGIPDYGFYTTPGDQRATLSFADYGSMGPRTAQMLRSEHAASASNSPLHHLPSPDQFKSPVLNSYSAQPNYGTPASPTGTNPGRPGGFPGANSPGPVADLYGTASQDSGVGNYISAASPQPGSGFSHGMAGPLIATAFTNGYH

SEQ ID NO: 17 - protein, 634 aa, Drosophila melanogaster
MLFENPAVAAKLPFPYNVPPPLQAAAAAAAAVPNLRFQTPIKAFACLTAATRSVSEMNATSLYAGNPMENAAAAAAAAAAGLIDPHHNRDLHQALVASIANNSVAAIGGGLTTAAVLKSAAQQSQQAVQQNQNAVVVTPGLEQPKQEPAQQAALALLKENVNASAGAGQNNGQAAMGGSNKSGSSGRSTPSLSGGSGSDPAPGKLFVGGLSWQTSSDKLKEYFNMFGTVTDVLIMKDPVTQRSRGFGFITFQEPCTVEKVLKVPIHTLDGKKIDPKHATPKNRPRQANKTKKIFVGGVSQDTSAEEVKAYFSQFGPVEETVMLMDQQTKRHRGFGFVTFENEDVVDRVCEIHFHTIKNKKVECKKAQPKEAVTPAAQLLQKRIMLGTLGVQLPTAPGQLIGARGAGVATMNPLAMLQNPTQLLQSPAAAAAAQQAALISQNPFQVQNAAAAASIANQAGFGKLLTTYPQTALHSVRYAPYSIPASAATANAALMQAHQAQSVAAAAHHHQQQQQQQHHHQQQTHNAHVAAAQQQQQSHHNAVSNPASQAHSAAAAAALAANAANGAGAAGAHSLAAAAQQAGLMAGNPLNAAAAAAAAAANPAAAYSNYALANVDMSSFQGVDWSTMYGMGMYV

(19) United States
(12) Patent Application Publication
(10) Pub. No.: US 2013/0011404 A1
(43) Pub. Date: Jan. 10, 2013

(54) Methods and Compositions for Regulating Musashi Function

(75) Inventors: Angus MacNicol, Little Rock, AR (US); Melanie MacNicol, Little Rock, AR (US); Karthik Arumugam, Little Rock, AR (US)

(73) Assignee: BOARD OF TRUSTEES OF THE UNIVERSITY OF ARKANSAS, Little Rock, AR (US)

(21) Appl. No.: 13/544,605

(22) Filed: Jul. 9, 2012

Related U.S. Application Data
(60) Provisional application No. 61/505,295, filed on Jul. 7, 2011.

(51) Int. Cl. C07K 16/18; C07K 14/435; G01N 33/566; G01N 21/76; A61K 38/02; A61K 31/7088; C12Q 1/68; C07H 21/04; A61K 39/395

(52) U.S. Cl. 424/139.1; 530/387.9; 536/23.5; 530/350; 436/501; 514/1.1; 514/44 R; 435/6.12; 435/8; 435/7.92

(57) ABSTRACT

The invention generally features compositions and methods for detecting and regulating cell proliferation, potentiation, and differentiation in a population of cells. In particular, compositions and methods are provided for modulating the activity of Musashi proteins. Diagnostic, screening, and therapeutic methods utilizing compositions of the invention are also provided.

### GOVERNMENT SUPPORT

This invention was made with government support under HD35688 and COBRE P20 (RR020146-01) awarded by the NIH. The government may have certain rights in the invention.

### FIELD OF THE INVENTION

The invention relates to compositions and methods for detecting and regulating normal and abnormal cell proliferation, potentiation, and differentiation in a population of cells. In particular, the invention relates to detecting and regulating the function of musashi proteins, which regulate the cell proliferation and differentiation of progenitor and stem cell types.

### BACKGROUND OF THE INVENTION

The Musashi mRNA-binding protein plays a critical role in the promotion of stem cell self-renewal by repressing the translation of mRNAs encoding proteins that inhibit cell cycle progression. Musashi was originally identified as a critical regulator of asymmetric cell division in Drosophila sensory organ precursor cells and subsequently, mammalian Musashi isoforms have been implicated in the self-renewal of neural, epithelial and hematopoietic stem and progenitor cells. Musashi has been also implicated in proliferative pathologies in various tissues where Musashi may be acting to promote self-renewal of tumor cells with stem cell-like properties. In this role, Musashi can act as an mRNA translational activator, as well as a translational repressor.

Two Musashi isoforms are presently known, Musashi1 and Musashi2. The Musashi proteins contain two N-terminal RNA recognition motifs (RRMs) that bind to target mRNAs through a Musashi binding element (MBE, (G/A)U₁₋₃AGU) in the mRNA 3′ untranslated region. The Musashi1 and Musashi2 isoforms are highly related in sequence within the RRM domains (>90% similarity at the amino acid level), suggesting they may interact with the same target mRNAs.

Musashi regulates a range of mRNAs encoding proteins involved in cell proliferation, cell differentiation, apoptosis, and protein modification (including ubiquitination). Specifically, Musashi proteins have been found to regulate proteins involved in inhibition of cell cycle progression, promote cell cycle exit and commitment of progenitor cells to differentiate. However, despite indications of a pivotal role in physiological and pathological stem cell proliferation, little is known about the mechanisms by which Musashi regulates mRNA translation or how Musashi function is regulated. Compositions and methods exploiting this pivotal role of Musashi function are needed to further medical research and provide diagnostic and therapeutic resouces for diseases associated with dysregulation of cell cycle homeostasis and stem cell regulation such as cancer.

### SUMMARY OF THE INVENTION

The Musashi proteins of the present invention, or biologically active portions thereof, can be operatively linked to a non-Musashi polypeptide (e.g., heterologous amino acid sequences) to form Musashi fusion proteins, respectively. The invention further features antibodies that specifically bind Musashi proteins, such as monoclonal or polyclonal antibodies. In addition, the Musashi proteins or biologically active portions thereof can be incorporated into pharmaceutical compositions, which optionally include pharmaceutically acceptable carriers.

In another aspect, the present invention provides a method for detecting the presence of Musashi activity or expression in a sample by contacting the sample with an agent capable of detecting an indicator of Musashi activity such that the presence of Musashi activity is detected in the biological sample.

In another aspect, the present invention provides a method for detecting the post-translational status of Musashi. In general, such methods include contacting a sample with an agent capable of detecting post-translational modifications of Musashi.

In another aspect, the invention provides a method for modulating Musashi activity comprising contacting a cell with an agent that modulates (inhibits or stimulates) Musashi activity or expression such that Musashi activity or expression in the cell is modulated. Examples of Musashi activity include the phosphorylation of Musashi. In one embodiment, the agent is an antibody that specifically binds to Musashi protein. In another embodiment, the agent modulates (increases or decreases) expression of Musashi by modulating transcription of a Musashi gene, splicing of a Musashi mRNA, or translation of a Musashi mRNA. In yet another embodiment, the agent is a nucleic acid molecule having a nucleotide sequence that is antisense to the coding strand of the Musashi mRNA or Musashi gene.

In one embodiment, the methods of the present invention are used to treat a subject having a disorder characterized by aberrant Musashi protein or nucleic acid expression or activity or related to Musashi expression or activity by administering an agent which is a Musashi modulator to the subject. In one embodiment, the Musashi modulator is a Musashi protein. In another embodiment the Musashi modulator is a Musashi nucleic acid molecule. In other embodiments, the Musashi modulator is a peptide, peptidomimetic, or other small molecule.

The present invention also provides a diagnostic assay for identifying the status of a population of cells. The status is dependent upon post-translational modification of Musashi protein.

In another aspect, the invention provides a method for identifying a compound that binds to or modulates the activity of a Musashi protein. In general, such methods entail measuring a biological activity of a Musashi protein in the presence and absence of a test compound and identifying those compounds which alter the activity of the Musashi protein.

In another aspect, the invention provides a method for identifying a compound that modulates (increases or decreases) the ability of Musashi to be post-translationally modified. In general, the method entails measuring the post-translational modification of Musashi in the presence and absence of a test compound or test compounds, and identifying the compound or compounds that modulate the post-translational modification of Musashi.

In another aspect, the invention provides a method for identifying a compound that modulates (increases or decreases) the ability of Musashi to stimulate cell proliferation or differentiation. In general, the method entails measuring the ability of Musashi to stimulate cell proliferation or differentiation in the presence and absence of a test compound or test compounds, and identifying the compound or compounds that modulate the ability of Musashi to stimulate the cell proliferation or differentiation.

The invention also features methods for identifying a compound which modulates the expression of Musashi by measuring the expression of Musashi in the presence and absence of a compound.

In another aspect, the invention provides a method of treating at least one symptom or sign of proliferative pathologies in a subject. In general, such methods include administering to a subject an effective amount of at least one Musashi agent.

### Reference to Color Figures

The application file contains at least one photograph executed in color. Copies of this patent application publication with color photographs will be provided by the Office upon request and payment of the necessary fee. The following drawings form part of the present specification and are included to further demonstrate certain aspects of the present invention. The invention may be better understood by reference to one or more of these drawings in combination with the detailed description of specific embodiments presented herein.

### BRIEF DESCRIPTION OF THE DRAWINGS

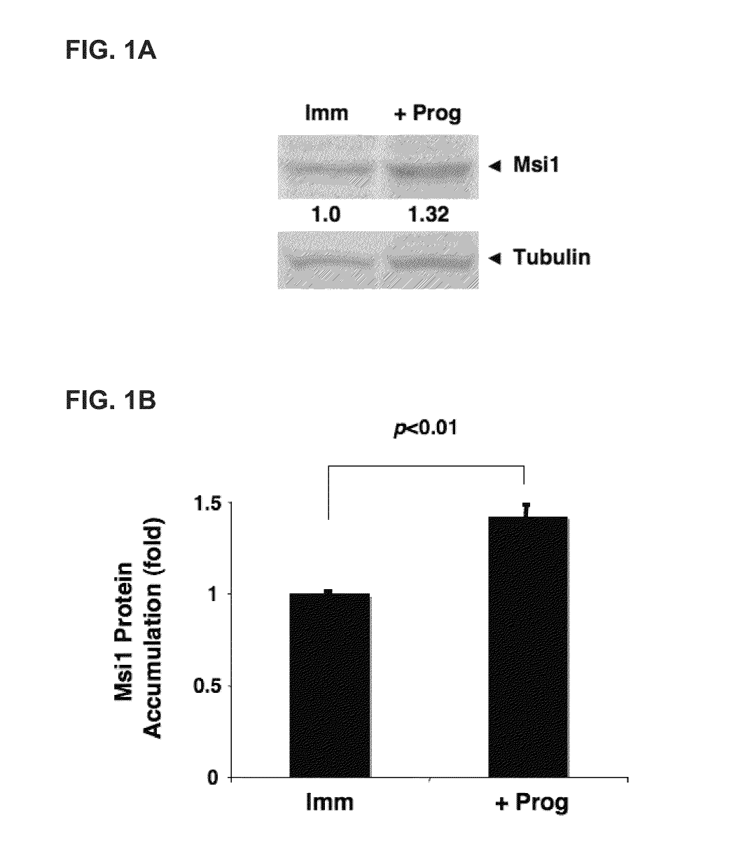

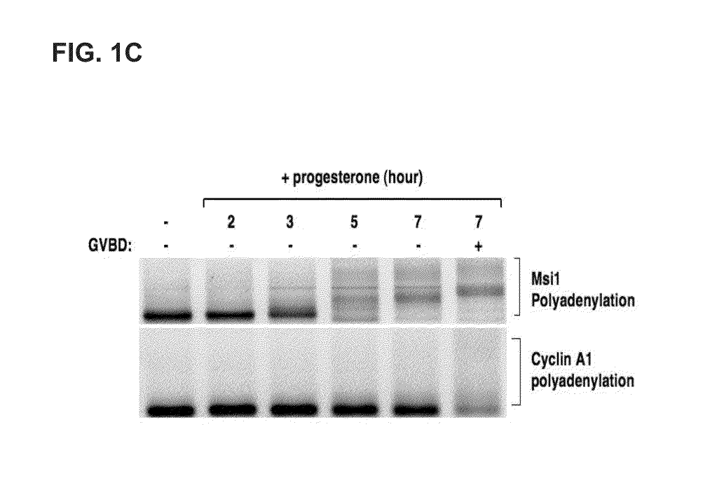

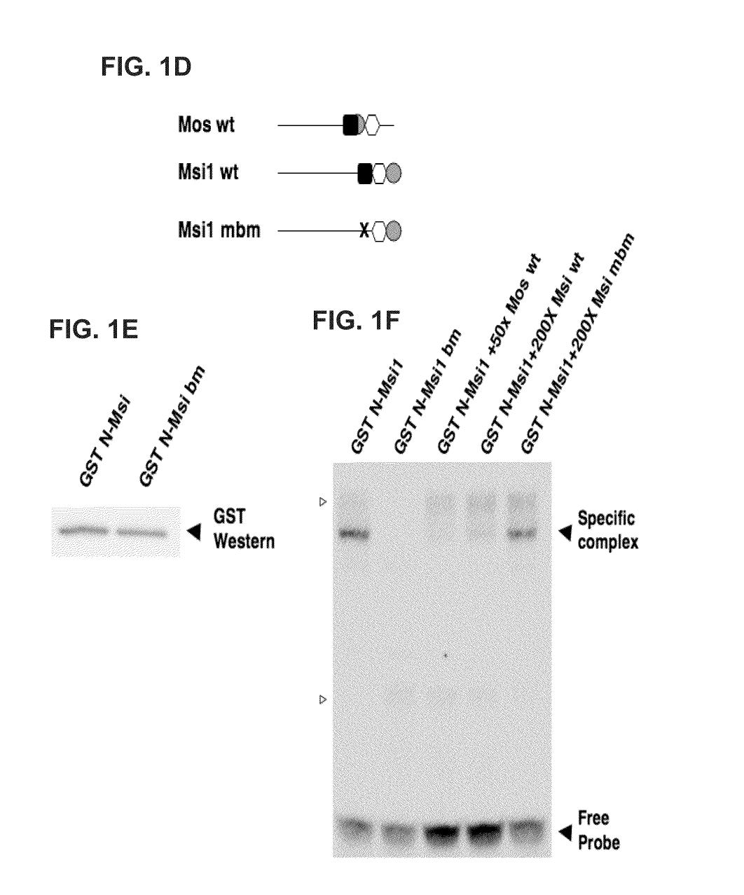

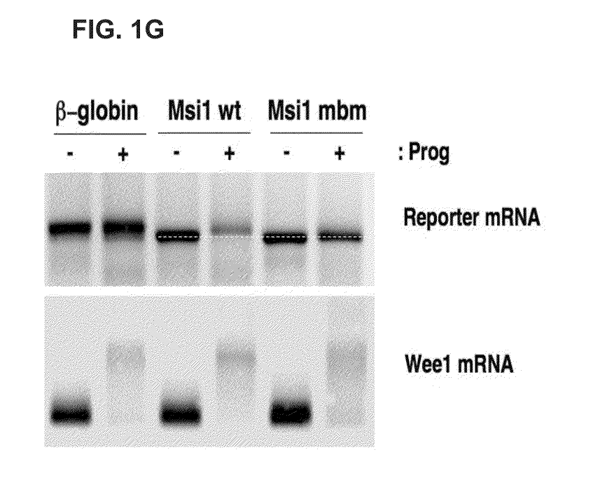

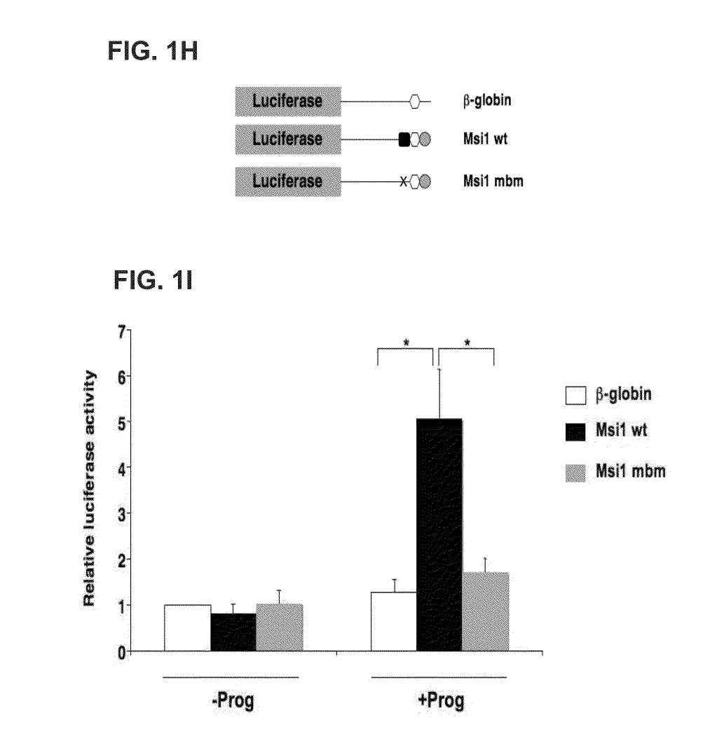

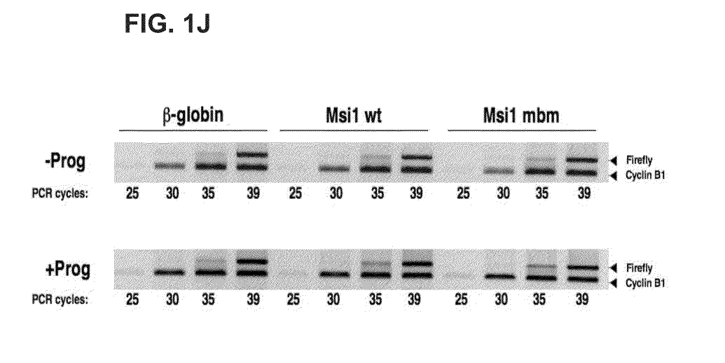

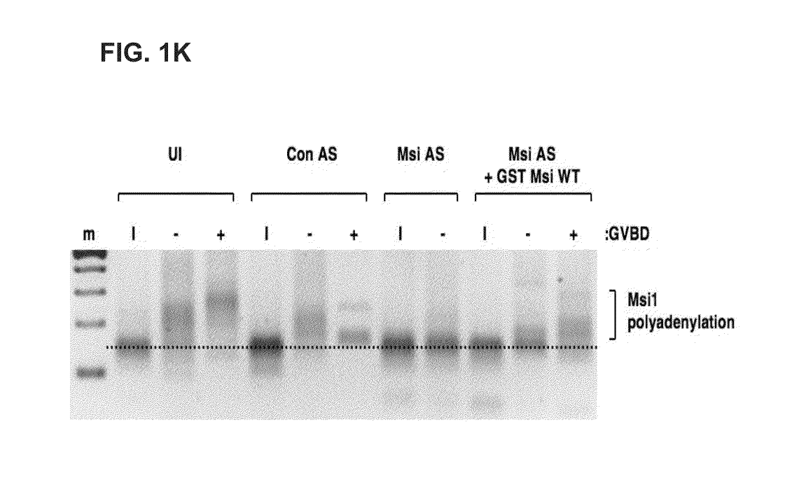

FIG. 1 shows that Musashi1 mRNA was activated during the early phase of oocyte maturation (FIGS. 1A and 1B) and undergoes polyadenylation early in maturation, prior to germinal vesicle nuclear breakdown (FIG. 1C). In particular, FIG. 1A depicts a western blot that shows Musashi protein levels increase in response to progesterone stimulation. FIG. 1B graphically illustrates a quantification of fold changes in Musashi1 protein levels in response to progesterone stimulation. FIG. 1C shows an RNA ligation-coupled PCR analysis that indicates Musashi mRNA is polyadenylated early in response to progesterone stimulation. Musashi protein was shown to interact specifically with a consensus Musashi Binding Element (MBE) found in the Musashi mRNA 3′ untranslated region (FIGS. 1D, 1E and 1F). FIG. 1D depicts a schematic of probe constructs used in the RNA-EMSA of FIG. 1F. The schematic shows the position of the polyadenylation hexanucleotide (hexagon), MBE (square) and CPE (shaded circle) for each construct. FIG. 1E depicts a western blot showing the N-terminal mRNA binding domain of Musashi! (N-Msi) or an RNA binding mutant form (N-Msi bm) were expressed as GST fusion protein in rabbit reticulocyte lysates for use in the EMSA reactions. FIG. 1F shows an RNA-EMSA analysis using the unlabeled RNA probes of FIG. 1D to compete Mos 3′ untranslated region (UTR) interaction with Musashi1 . The progesterone-stimulated polyadenylation of the Musashi mRNA was dependent upon the MBE (FIG. 1G), as was translation of an mRNA reporter linked to the Musashi1 3′ untranslated region (FIGS. 1H and 1I). FIG. 1G shows an RNA-ligation-coupled PCR analysis showing that the wild-type xMsi1 3′UTR was polyadenylated upon progesterone stimulation (retarded mobility of the PCR product relative to that present in immature oocytes as observed above the dashed reference line). Polyadenylation directed by the Musashi1 3′UTR was completely abrogated when the MBE Was disrupted (Msi mbm). No progesterone-dependent polyadenylation of a control reporter under the control of the unregulated β-globin 3′UTR was observed. FIG. 1H shows a schematic representation of the 3′ UTR firefly luciferase reporter constructs utilized with consensus Musashi binding element (MBE, black square), consensus cytoplasmic polyadenylation element (CPE, white circle) and the consensus polyadenylation hexanucleotide (gray hexagon) indicated. The disrupted MBE IN the Musashi binding mutant (mbm) UTR is shown as an “x”. FIG. 11 graphically depicts a plot showing an average ratio of firefly luciferase activity for the Musashi1 reporters relative to co-injected Renilla luciferase from three independent experiments. The stability of the reporter constructs is shown in FIG. 1J in response to progesterone. FIG. 1K depicts an RNA ligation-coupled PCR analysis that shows translational activation of endogenous Musashi1 mRNA is mediated by Musashi (UI, uninjected, no antisense injection; Con AS, control antisense injection; Msi AS, Musashi antisense, no rescue; I, immature oocytes, no progesterone.

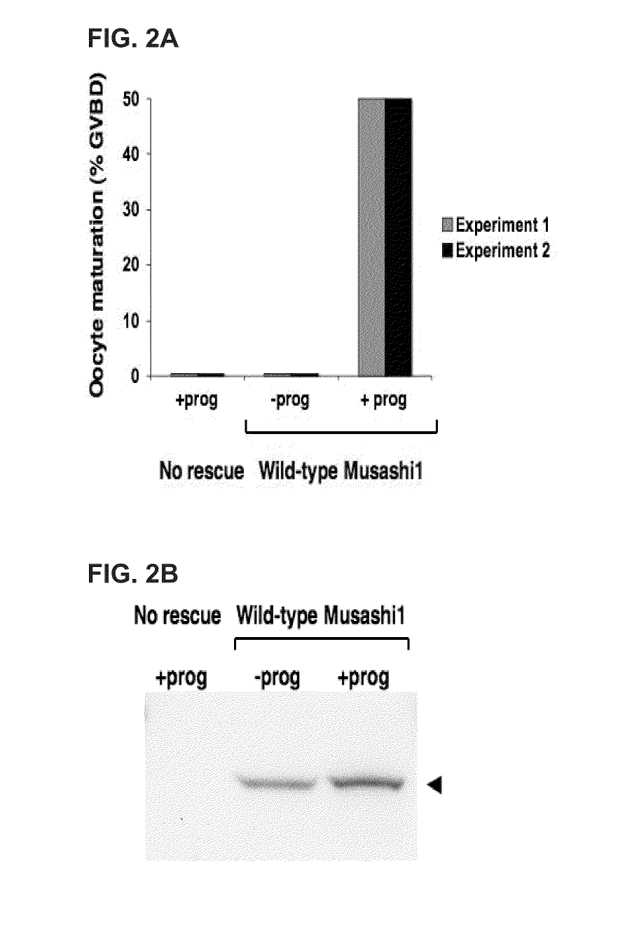

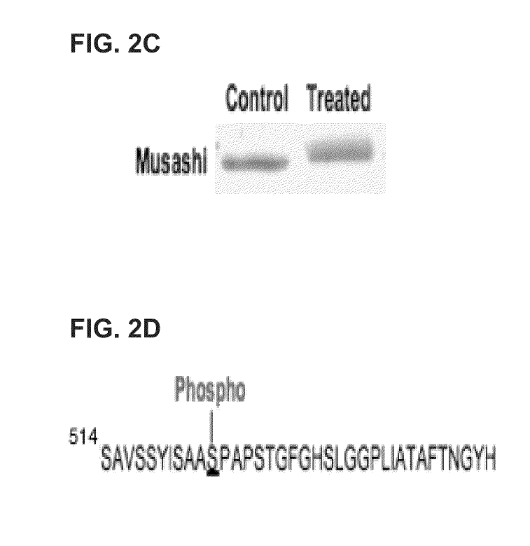

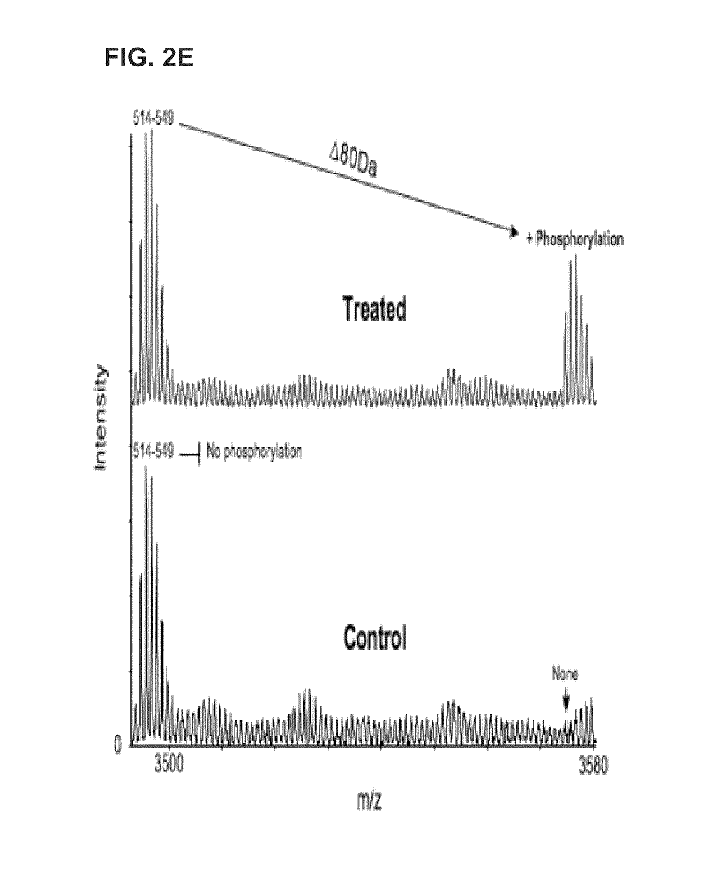

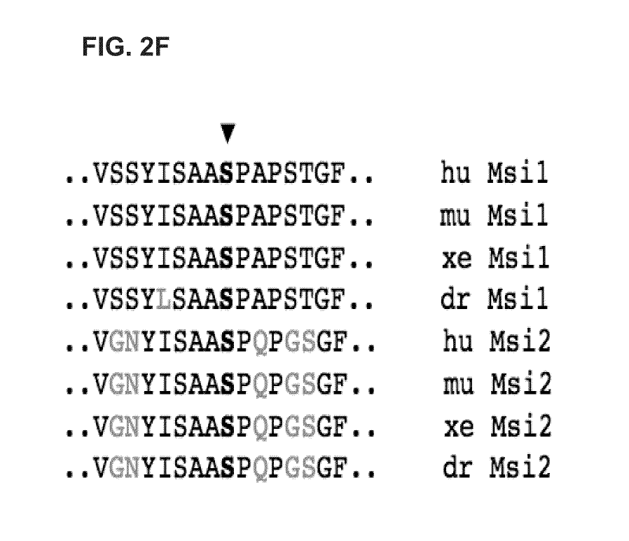

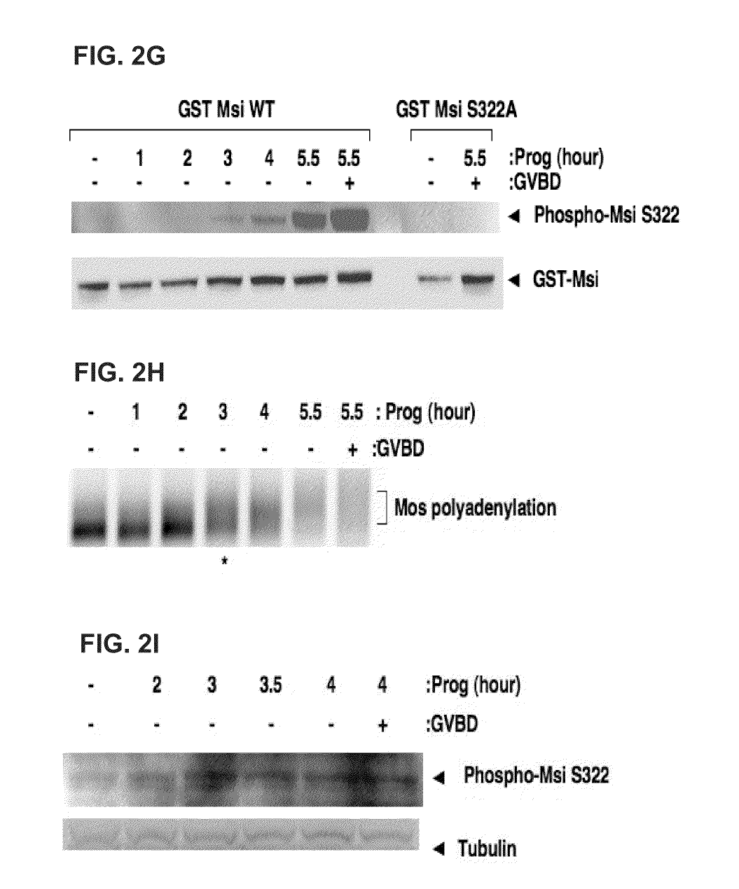

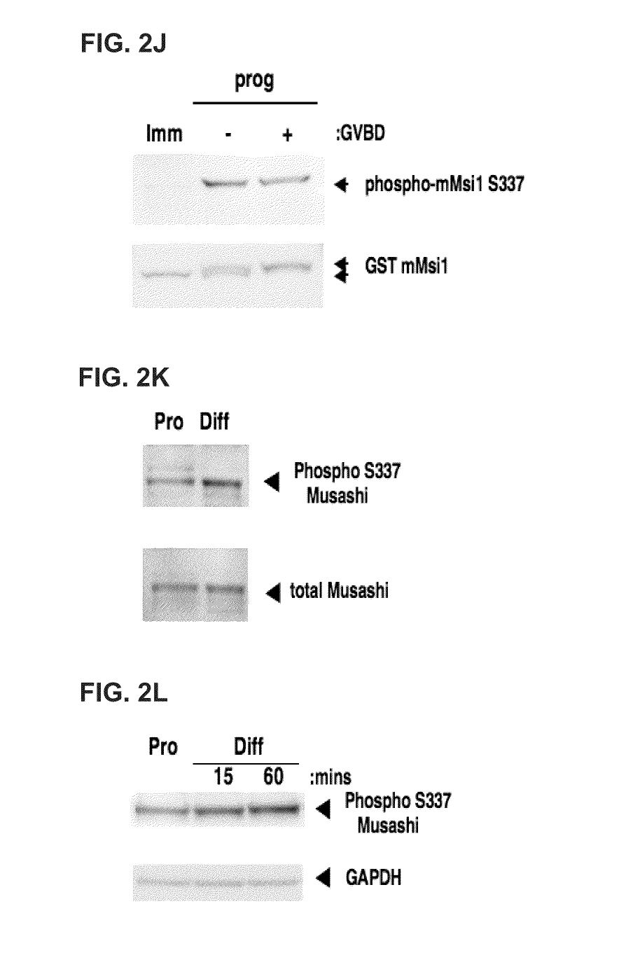

FIG. 2 illustrates that Musashi1 undergoes stimulus-dependent phosphorylation at a conserved serine amino acid residue. FIG. 2A graphically illustrates a quantification of oocyte maturation in response to antisense DNA oligonucleotides targeting Musashi1and Musashi2 in the presence or absence of progesterone and rescued with wild-type Musashi1. FIG. 2B shows a GST western blot confirming the expression of ectopic Musashi1 protein in FIG. 2A. Over-expression of Musashi was not sufficient to induce maturation in the absence of progesterone stimulation (FIGS. 2A and 2B). FIG. 2C shows a Coomassie-stained gel of GST-Musashi1 protein partially purified from immature and mature oocytes. Gel bands were excised and subjected to mass spectrometry analysis. FIG. 2E shows an expanded view of the MALDI spectra that indicate exclusive phosphorylation (+80 Da) of peptide 514-549 in the treated sample shown in FIG. 2C. MALDI MS² and MS³ analyses (vMALDI-LTQ, Thermo) were used to site-specifically map the site of phosphorylation to Ser524 of the GST-Musashi1 protein, corresponding to Ser322 of the Musashi protein without the GST epitope tag. FIG. 2D depicts the amino acid sequence surrounding the phosphorylation site at Ser322. FIG. 2F shows the phosphorylation site and surrounding amino acids are conserved among several species. FIG. 2G shows a western blot of oocyte lysates with antibody specific to the S322 phosphorylated form of Musashi1 that demonstrates progesterone-stimulated phosphorylation is not observed in the S322A mutant Musashi. A GST western blot shows equivalent levels of the expressed proteins (FIG. 2G, lower panel). FIG. 2H shows a polyadenylation assay that indicates the Musashi mRNA target, Mos, is activated coincident with Musashi1 S322 phosphorylation. FIG. 21 depicts a western blot that shows endogenous Musashi1 is phosphorylated on S322 in response to progesterone stimulation. FIG. 2J depicts a western blot probed with phosphor S322 Musashi1 specific antiserum and GST antiserum which shows mammalian Musashi1 is phosphorylated on S337 in response to progesterone stimulation. FIG. 2K depicts a western blot that shows Musashi1 is phosphorylated on S337 in differentiating embryonic rat neural stem/progenitor cells. FIG. 2L depicts a western blot that shows Musashi1 is phosphorylated on S337 in differentiating SH-SY5Y neuroblastoma cells.

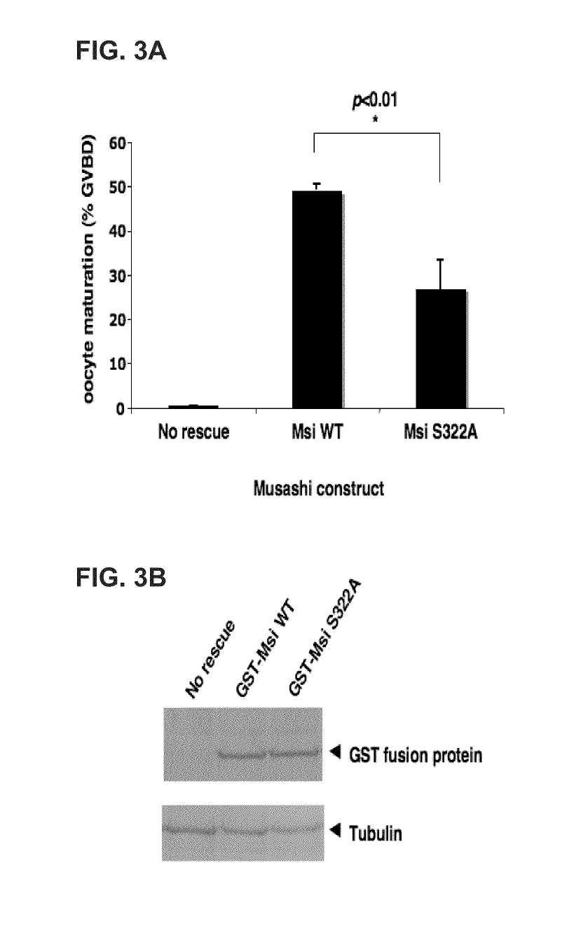

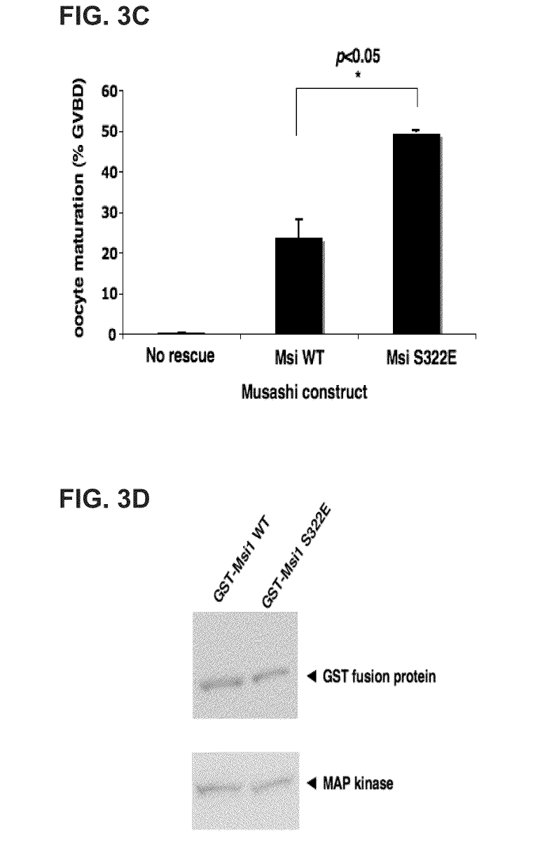

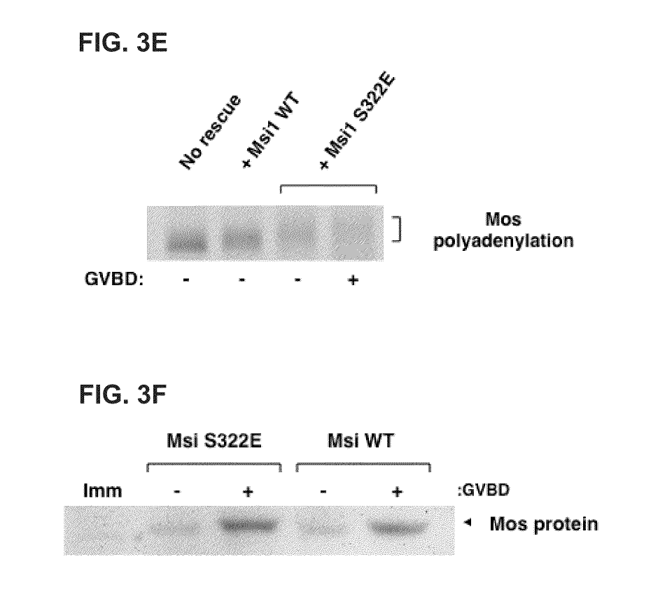

FIG. 3 shows Musashi1 phosphorylation facilitates oocyte maturation and target mRNA translational activation. FIG. 3A graphically depicts the quantification of oocyte maturation showing the inhibition of Musashi1 S322 phosphorylation attenuates oocyte maturation. FIG. 3B depicts a western blot showing that Musashi (Msi) wild-type and S322A proteins were expressed to equivalent levels in the rescue assay of FIG. 3A. FIG. 3C graphically depicts the quantification of oocyte maturation that shows that mutational mimicry of Musashi1 S322 phosphorylation accelerates oocyte maturation. FIG. 3D depicts a western blot that shows the Musashi wild-type and S322E proteins were expressed to equivalent levels in the rescue as of FIG. 3C. FIG. 3E shows a polyadenylation assay that indicates mutational mimicry of Musashi1 S322 phosphorylation accelerates activation of the Mos mRNA. FIG. 3F depicts a western blot that shows mutational mimicry of Musashi S322 phosphorylation enhances mos protein accumulation.

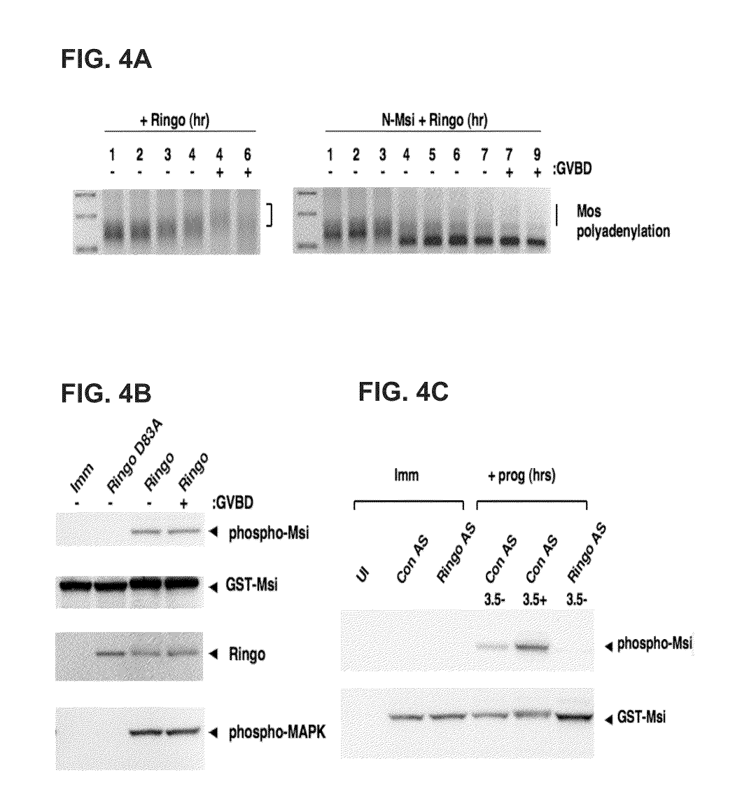

FIG. 4 shows Musashi function is necessary for Ringo-induced early class mRNA translational activation. FIG. 4A shows a polyadenylation assay that indicates that in the N-Msi expressing oocytes, Ringo induced deadenylation of the Mos mRNA. FIG. 4B is a western blot that shows Musashi function is necessary for Ringo-induced early class mRNA translational activation. FIG. 4C is a western blot that shows there was no maturation of Ringo antisense injected oocytes.

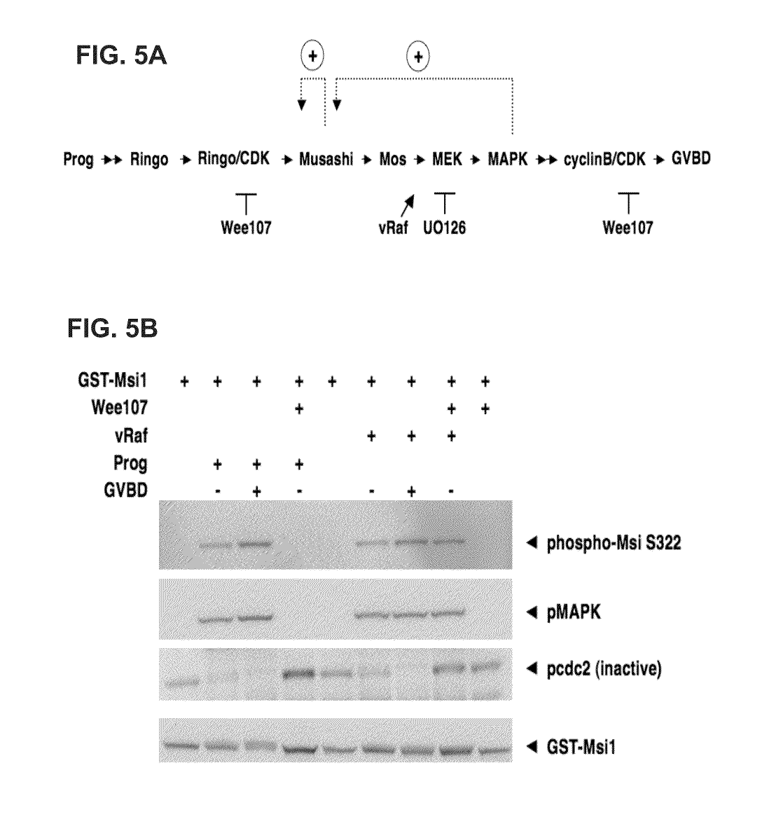

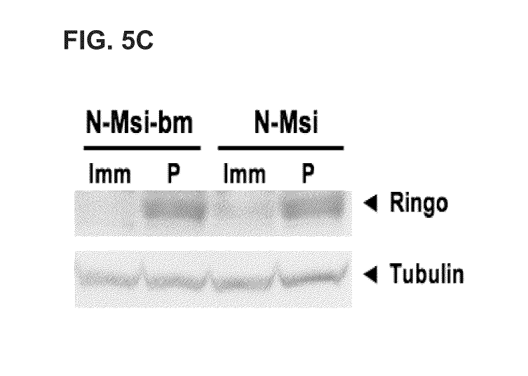

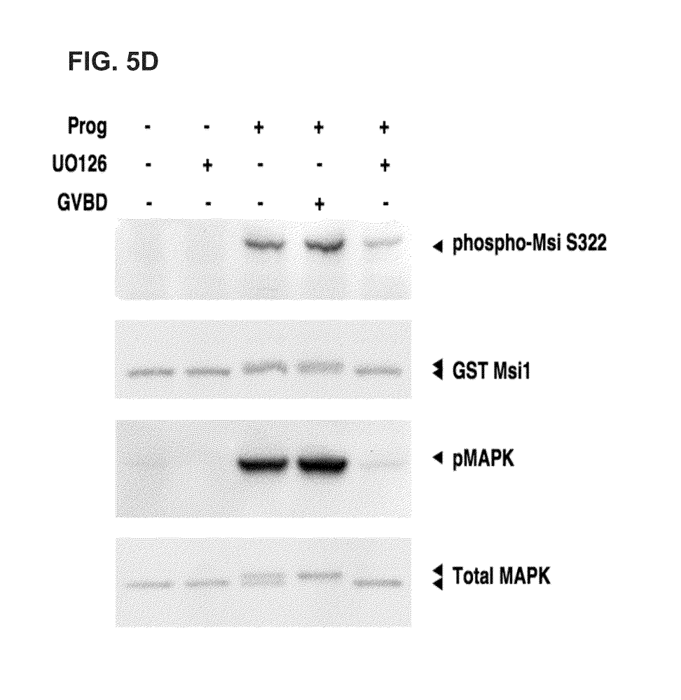

FIG. 5 shows that Ringo/CDK and MAP kinase direct Musashi phosphorylation on S322. FIG. 5A shows a schematic illustrating relevant progesterone-dependent signaling events impinging upon early class Musashi-mediated, mRNA translation prior to MPF (cyclin B/CDK) activation and oocyte GVBD. The points of experimental manipulation are shown along with the deduced Musashi amplification loops. FIG. 5B is a western blot that shows MAP kinase signaling induces Musashi1 S322 phosphorylation independently of CDK. FIG. 5C is a western blot showing that the expression of a dominant inhibitory form of Musashi does not block progesterone-stimulated Ringo accumulation. FIG. 5D is a western blot showing that progesterone-stimulated Musashi1 S322 phosphorylation is mediated by both MAP kinase-dependent and MAP kinase-independent signaling.

### DESCRIPTION OF PREFERRED EMBODIMENTS

Applicants have discovered mechanisms necessary for the regulation of a protein that plays a pivotal role in regulating cell proliferation and differentiation of progenitor cells including adult, embryonic, and cancer stem cell types. The present invention encompasses this discovery and provides compositions and methods based on the discovered regulatory mechanism. In particular, the present invention provides compositions and methods useful in research, diagnostics, and therapeutics for conditions and diseases associated with pathologic proliferation or differentiation. The compositions and methods are directed at detecting and regulation the post-translational status of Musashi protein family members.

Various aspects of the invention are described in further detail in the following subsections.

### I. Compositions

A. Nucleic Acid Molecules

One aspect of the invention pertains to isolated nucleic acid molecules that encode Musashi proteins or biologically active portions thereof, as well as nucleic acid molecules sufficient for use as hybridization probes to identify Musashi-encoding nucleic acids (e.g., Musashi mRNA) and fragments for use as PCR primers for the amplification or mutation of Musashi nucleic acid molecules.

A nucleic acid molecule of the present invention, e.g., a nucleic acid molecule having the nucleotide sequence of SEQ ID NO:1-7, or a complement of any of these nucleotide sequences, may be isolated using standard molecular biology techniques and the sequence information provided herein. Using all or portion of the nucleic acid sequences of SEQ ID NO:1-7, Musashi nucleic acid molecules may be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention may be amplified using cDNA, mRNA or genomic DNA as a template and appropriate oligonucleotide primers according to standard PCR amplification techniques. The nucleic acid so amplified may be cloned into an appropriate vector and characterized by DNA sequence analysis. Furthermore, oligonucleotides corresponding to Musashi nucleotide sequences may be prepared by standard synthetic techniques known in the art, such as using an automated DNA synthesizer.

In another embodiment, an isolated nucleic acid molecule of the invention comprises a nucleic acid molecule which is a complement of the nucleotide sequence shown in SEQ ID NO:1-7, or portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, the nucleic acid molecule of the invention may comprise only a portion of a nucleic acid sequence encoding Musashi. By way of example, a fragment of the nucleic acid coding sequence can be used as a probe, primer, or a fragment encoding a biologically active portion of Musashi. The nucleotide sequence determined from the cloning of the Musashi gene allows for the generation of probes and primers designed for use in identifying and/or cloning Musashi homologues in other cell types, as well as Musashi homologues and orthologs from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or antisense sequence of SEQ ID NO:1-7, or of a naturally occurring mutant of one of SEQ ID NO:1-7.

Probes based on the Musashi nucleotide sequence may be used to detect transcripts or genomic sequences encoding the same or similar proteins. The probe comprises a label group attached thereto, such as a radioisotope, a fluorescent compound, an enzyme, or an enzyme co-factor. Such probes may be used in diagnostic or screening assays.

A nucleic acid fragment encoding a “biologically active portion” of Musashi may be prepared by isolating a portion of SEQ ID NO:1-7, which encodes a polypeptide having a Musashi biological activity, expressing the encoded portion of Musashi protein (e.g., by recombinant expression in vitro) and assessing the activity of the encoded portion of Musashi. For example, a nucleic acid fragment encoding a biologically active portion of Musashi includes a post-translational modification site, or an RNA binding site.

The invention further encompasses nucleic acid molecules that differ from the nucleotide sequence of SEQ ID NO:1-7, due to degeneracy of the genetic code and thus encode the same Musashi protein as that encoded by the nucleotide sequence shown in SEQ ID NO:1-7.

In addition to the Musashi nucleotide sequence shown in SEQ ID NO:1-7, it will be appreciated by those skilled in the art that DNA sequence polymorphisms that lead to changes in the amino acid sequences of Musashi may exist within a population (e.g., the human population). Such genetic polymorphism in the Musashi gene may exist among individuals within a population due to natural allelic variation. Such natural allelic variations typically result in 15% variance in the nucleotide sequence of the Musashi gene. Any and all such nucleotide variations and resulting amino acid polymorphisms in Musashi that are the result of natural allelic variation and that do not alter the functional activity of Musashi are intended to be within the scope of the invention. Thus, e.g., 1%, 2%, 3%, 4%, or 5% of the amino acids in Musashi (e.g., 1, 2, 3, 4, 5, 6, 8, 10, 15, 20, 25, 30, 35, 40, 45, 50, or 55 amino acids) may be replaced by another amino acid, preferably by conservative substitution.

Moreover, nucleic acid molecules encoding Musashi proteins from other species (Musashi orthologs/homologues), which have a nucleotide sequence which differs from that of a Musashi disclosed herein, are intended to be within the scope of the invention.

Accordingly, in another embodiment, an isolated nucleic acid molecule of the invention is at least 150 (300, 325, 350, 375, 400, 425, 450, 500, 550, 600, 650, 700, 800, 900, 1000, 1200, 1400, 1600, 1800, 2000, 2200, 2400, 2600, 2800, 3000, 3200, 3400, 3600, 3800, or 3900) nucleotides in length and hybridizes under stringent conditions to the nucleic acid molecule comprising the nucleotide sequence, preferably the coding sequence, of SEQ ID NO:1-7.

In addition to naturally occurring allelic variants of the Musashi sequence that may exist in the population, the skilled artisan will further appreciate that changes may be introduced by mutation into the nucleotide sequence of SEQ ID NO:1-7, thereby leading to changes in the amino acid sequence of the encoded protein without altering the functional ability of the protein. For example, such mutations may include nucleotide substitutions leading to amino acid substitutions at “nonessential” amino acid residues. A “nonessential” amino acid residue is a residue that may be altered from the wildtype sequence of Musashi protein without altering the biological activity, whereas an “essential” amino acid residue is required for biological activity.

Accordingly, another aspect of the invention pertains to nucleic acid molecules encoding Musashi proteins that contain changes in amino acid residues that may or may not be essential for activity. Such Musashi proteins differ in amino acid sequence from SEQ ID NO:11-17. In one embodiment, the isolated nucleic acid molecule includes a nucleotide sequence encoding a protein that includes an amino acid sequence that is at least about 45% identical, 65%, 75%, 85%, 95%, or 98% identical to the amino acid sequence of SEQ ID NO:11-17. An isolated nucleic acid molecule encoding a Musashi protein having a sequence which differs from that of SEQ ID NO:1-7, may be created by introducing one or more nucleotide substitutions, additions or deletions into the nucleotide sequence of Musashi (SEQ ID NO:1-7) such that one or more amino acid substitutions, additions or deletions are introduced into the encoded protein. Mutations may be introduced by standard techniques known in the art, such as site-directed mutagenesis and PCR-mediated mutagenesis.

The present invention encompasses antisense nucleic acid molecules. Antisense molecules are complementary to a sense nucleic acid encoding a protein, complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire Musashi coding strand, or to only a portion thereof, such as all or part of the protein coding region (or open reading frame). An antisense nucleic acid molecule can be antisense to a noncoding region of the coding strand of a nucleotide sequence encoding Musashi. The noncoding regions (“5′ and 3′ untranslated regions”) are the 5′ and 3′ sequences that flank the coding region and are not translated into amino acids. Given the coding strand sequences encoding Musashi disclosed herein, antisense nucleic acids of the invention may be designed according to the rules of Watson and Crick base pairing. The antisense nucleic acid molecule may be complementary to the entire coding region of Musashi mRNA, but more preferably is an oligonucleotide which is antisense to only a portion of the coding or noncoding region of Musashi mRNA. For example, the antisense oligonucleotide may be complementary to the region surrounding the translation start site of Musashi mRNA. An antisense oligonucleotide may be, for example, about 5, 10, 15, 20, 25, 30, 35, 40, 45 or 50 nucleotides in length. An antisense nucleic acid of the invention may be constructed using chemical synthesis and enzymatic ligation reactions using procedures known in the art. For example, an antisense nucleic acid (e.g., an antisense oligonucleotide) may be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which may be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-aino-3-N-2-carboxypropyl) uracil, (acp3)w, and 2,6-diaminopurine. Alternatively, the antisense nucleic acid may be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a Musashi protein to thereby inhibit expression of the protein, e.g., by inhibiting transcription and/or translation. The hybridization may be by conventional nucleotide complementarity to form a stable duplex, or, for example, in the case of an antisense nucleic acid molecule which binds to DNA duplexes, through specific interactions in the major groove of the double helix. An antisense nucleic acid molecule of the invention may be administered by direct injection at a tissue site. Alternatively, antisense nucleic acid molecules may be modified to target selected cells and then administered systemically. For example, for systemic administration, antisense molecules may be modified such that they specifically bind to receptors or antigens expressed on a selected cell surface, e.g., by linking the antisense nucleic acid molecules to peptides or antibodies which bind to cell surface receptors or antigens. The antisense nucleic acid molecules may also be delivered to cells using the plasmids described herein. To achieve sufficient intracellular concentrations of the antisense molecules, plasmid constructs in which the antisense nucleic acid molecule is placed under the control of a strong pol II or pol III promoter are preferred.

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) may be used to catalytically cleave Musashi mRNA transcripts to thereby inhibit translation of Musashi mRNA. A ribozyme having specificity for a Musashi-encoding nucleic acid may be designed based upon the nucleotide sequence of a Musashi cDNA disclosed herein. For example, Musashi mRNA may be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, Musashi gene expression may be inhibited by targeting nucleotide sequences complementary to the regulatory region of Musashi (e.g., the Musashi promoter and/or enhancers) to form triple helical structures that prevent transcription of the Musashi gene in target cells. See generally, Helene (1991) Anticancer Drug Des. 6(6):569-84; Helene (1992) Ann. N.Y. Acad. Sci. 660:27-36; and Maher (1992) Bioassays 14(12):807-15.

In embodiments, the nucleic acid molecules of the invention may be modified at the base moiety, sugar moiety or phosphate backbone to improve, e.g., the stability, hybridization, or solubility of the molecule. For example, the deoxyribose phosphate backbone of the nucleic acids may be modified to generate peptide nucleic acids (see Hyrup et al. (1996) Bioorganic & Medicinal Chemistry 40:5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers may be performed using standard solid phase peptide synthesis protocols as described in Hyrup et al. (1996) supra; Perry-O′Keefe et al. (1996) Proc. Natl. Acad. Sci. USA 93:14670-675.

PNAs of Musashi may be used for therapeutic and diagnostic applications. For example, PNAs may be used as antisense or antigene agents for sequence-specific modulation of gene expression by inducing transcription or translation arrest or inhibiting replication. PNAs of Musashi may also be used in the analysis of single base pair mutations in a gene by PNA directed PCR clamping; as artificial restriction enzymes when used in combination with other enzymes, such as 51 nucleases (Hyrup (1996) supra) or as probes or primers for DNA sequence and hybridization (Hyrup (1996) supra; Perry-O'Keefe et al. (1996) Proc. Natl. Acad. Sci. USA 93: 14670-675).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides may be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

B. Musashi Proteins

One aspect of the invention pertains to isolated Musashi proteins, and biologically active portions thereof, as well as polypeptide fragments suitable for use as immunogens to raise anti-Musashi antibodies. In one embodiment, native Musashi proteins may be isolated from cells or tissue sources by an appropriate purification scheme using standard protein purification techniques. In another embodiment, Musashi proteins are produced by recombinant DNA techniques. Alternative to recombinant expression, a Musashi protein or polypeptide may be synthesized chemically using standard peptide synthesis techniques.

An “isolated” or “purified” protein or biologically active portion thereof is substantially free of cellular material or other contaminating proteins from the cell or tissue source from which the Musashi protein is derived, or substantially free from chemical precursors or other chemicals when chemically synthesized. The language “substantially free of cellular material” includes preparations of Musashi protein in which the protein is separated from cellular components of the cells from which it is isolated or recombinantly produced. Thus, Musashi protein that is substantially free of cellular material includes preparations of Musashi protein having less than about 30%, 20%, 10%, or 5% (by dry weight) of non-Musashi protein (also referred to herein as a “contaminating protein”). When the Musashi protein or biologically active portion thereof is recombinantly produced, it is also preferably substantially free of culture medium, i.e., culture medium represents less than about 20%, 10%, or 5% of the volume of the protein preparation. When Musashi protein is produced by chemical synthesis, it is preferably substantially free of chemical precursors or other chemicals, i.e., it is separated from chemical precursors or other chemicals which are involved in the synthesis of the protein. Accordingly, such preparations of Musashi protein have less than about 30%, 20%, 10%, 5% (by dry weight) of chemical precursors or non-Musashi chemicals.

Biologically active portions of a Musashi protein include peptides comprising amino acid sequences sufficiently identical to or derived from the amino acid sequence of the Musashi protein (e.g., the amino acid sequence shown in SEQ ID NO:11-17), which include less amino acids than the full length Musashi protein, and exhibit at least one activity of a Musashi protein. Typically, biologically active portions comprise a domain or motif with at least one activity of the Musashi protein. A biologically active portion of a Musashi protein may be a polypeptide which is, for example, 10, 25, 50, 100, 150, 200, 250, 300 or more amino acids in length. Preferred biologically active polypeptides include one or more identified Musashi structural domains, such as RNA binding domains, phosphaylation sites, translational repression domain, translational activation domains, subcellular localization domain (nuclear localization sequence) and other domains that may be discovered.

Moreover, other biologically active portions, in which other regions of the protein are deleted, may be prepared by recombinant techniques and evaluated for one or more of the functional activities of a native Musashi protein.

The Musashi protein has the amino acid sequence of SEQ ID NO:11-17. Other useful Musashi proteins are substantially identical to SEQ ID NO:11-17 and retain the functional activity of the protein of SEQ ID NO:11-17, yet differ in amino acid sequence due to natural allelic variation or mutagenesis.

A useful Musashi protein is a protein which includes an amino acid sequence at least about 45%, preferably 55%, 65%, 75%, 85%, 95%, or 99% identical to the amino acid sequence of SEQ ID NO:11-17, and retains the functional activity of the Musashi protein of SEQ ID NO:11-17.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions.times.100).

The determination of percent homology between two sequences may be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences similar or homologous to Musashi nucleic acid molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used.

The invention also provides Musashi chimeric or fusion proteins. As used herein, a Musashi “chimeric protein” or “fusion protein” comprises a Musashi polypeptide operatively linked to a non-Musashi polypeptide. A “Musashi polypeptide” refers to a polypeptide having an amino acid sequence corresponding to all or a portion (preferably a biologically active portion) of a Musashi, whereas a “non-Musashi polypeptide” refers to a polypeptide having an amino acid sequence corresponding to a protein which is not substantially identical to a Musashi protein. Within the fusion protein, the term “operatively linked” is intended to indicate that the Musashi polypeptide and the non-Musashi polypeptide are fused in-frame to each other. The heterologous polypeptide may be fused to the N-terminus or C-terminus of the Musashi polypeptide.

One useful fusion protein is a GST fusion protein in which the Musashi sequences are fused to the C-terminus of the GST sequences. Such fusion proteins can facilitate the purification of recombinant Musashi.

In yet another embodiment, the fusion protein is a Musashi-immunoglobulin fusion protein in which all or part of Musashi is fused to sequences derived from a member of the immunoglobulin protein family. The Musashi-immunoglobulin fusion proteins of the invention may be incorporated into pharmaceutical compositions and administered to a subject to inhibit an interaction between a Musashi target mRNA and a Musashi protein to thereby suppress Musashi activity in vivo. The Musashi-immunoglobulin fusion proteins may be used to affect the bioavailability of a Musashi target mRNA. Inhibition of the Musashi target mRNA/Musashi interaction may be useful therapeutically for both the treatment of proliferative and differentiative disorders, as well as modulating (e.g., promoting or inhibiting) cell survival. Moreover, the Musashi-immunoglobulin fusion proteins of the invention may be used as immunogens to produce anti-Musashi antibodies in a subject, to purify Musashi ligands and in screening assays to identify molecules which inhibit the interaction of Musashi with a Musashi target mRNA.

Preferably, a Musashi chimeric or fusion protein of the invention is produced by standard recombinant DNA techniques. For example, DNA fragments coding for the different polypeptide sequences are ligated together in-frame in accordance with conventional techniques. Suitable techniques include by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A Musashi-encoding nucleic acid may be cloned into such an expression vector such that the fusion moiety is linked in-frame to the Musashi protein.

The present invention also pertains to variants of the Musashi proteins which function as either Musashi agonists (mimetics) or as Musashi antagonists. Variants of the Musashi proteins may be generated by mutagenesis techniques known in the art. An agonist of the Musashi protein may retain substantially the same, or a subset, of the biological activities of the naturally occurring form of the Musashi protein. An antagonist of the Musashi protein may inhibit one or more of the activities of the naturally occurring form of the Musashi protein by, for example, competitively binding to a downstream or upstream member of a cellular signaling cascade which includes the Musashi protein, or by inhibiting binding to target mRNAs. Thus, specific biological effects may be elicited by treatment with a variant of limited function. Treatment of a subject with a variant having a subset of the biological activities of the naturally occurring form of the protein may have fewer side effects in a subject relative to treatment with the naturally occurring form of the Musashi proteins.

Variants of the Musashi protein which function as either Musashi agonists (mimetics) or as Musashi antagonists can be identified by screening combinatorial libraries of mutants, such as truncation mutants of the Musashi protein for Musashi protein agonist or antagonist activity. In one embodiment, a variegated library of Musashi variants is generated by combinatorial mutagenesis at the nucleic acid level and is encoded by a variegated gene library. A variegated library of Musashi variants may be produced by, for example, enzymatically ligating a mixture of synthetic oligonucleotides into gene sequences such that a degenerate set of potential Musashi sequences is expressible as individual polypeptides, or alternatively, as a set of larger fusion proteins (e.g., for phage display) containing the set of Musashi sequences therein. There are a variety of methods which may be used to produce libraries of potential Musashi variants from a degenerate oligonucleotide sequence. Chemical synthesis of a degenerate gene sequence may be performed in an automatic DNA synthesizer, and the synthetic gene then ligated into an appropriate expression vector. Use of a degenerate set of genes allows for the provision, in one mixture, of all of the sequences encoding the desired set of potential Musashi sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the Musashi protein coding sequence can be used to generate a variegated population of Musashi fragments for screening and subsequent selection of variants of a Musashi protein. In one embodiment, a library of coding sequence fragments can be generated by treating a double stranded PCR fragment of a Musashi coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with 51 nuclease, and ligating the resulting fragment library into an expression vector. By this method, an expression library can be derived which encodes N-terminal and internal fragments of various sizes of the Musashi protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of Musashi proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify Musashi variants (Arkin and Yourvan (1992) Proc. Natl. Acad. Sci. USA 89:7811-7815; Delgrave et al. (1993) Protein Engineering 6(3):327-331).

C. Antibodies

An isolated Musashi protein, or a portion or fragment thereof, can be used as an immunogen to generate antibodies that bind Musashi using standard techniques for polyclonal and monoclonal antibody preparation. The full-length Musashi protein can be used or, alternatively, the invention provides antigenic peptide fragments of Musashi for use as immunogens. The antigenic peptide of Musashi comprises at least 8 (preferably 10, 15, 20, or 30) amino acid residues of the amino acid sequence shown in SEQ ID NO:1-7 and encompasses an epitope of Musashi such that an antibody raised against the peptide forms a specific immune complex with Musashi. Preferably, the antigenic peptide of Musashi comprises at least 8 or more amino acids of sequences shown in SEQ ID NO: 8-10.

Useful antibodies include antibodies which bind to a domain or subdomain of Musashi described herein (e.g., a RNA binding domain or a post-translational modification site).

A Musashi immunogen typically is used to prepare antibodies by immunizing a suitable subject, (e.g., rabbit, goat, mouse or other mammal) with the immunogen. An appropriate immunogenic preparation may contain, for example, recombinantly expressed Musashi protein or a chemically synthesized Musashi polypeptide. The preparation may further include an adjuvant, such as Freund's complete or incomplete adjuvant, or similar immunostimulatory agent. Immunization of a suitable subject with an immunogenic Musashi preparation induces a polyclonal anti-Musashi antibody response.

Accordingly, another aspect of the invention pertains to anti-Musashi antibodies. The term “antibody” as used herein refers to immunoglobulin molecules and immunologically active portions of immunoglobulin molecules, i.e., molecules that contain an antigen binding site which specifically binds an antigen, such as Musashi. A molecule which specifically binds to Musashi is a molecule which binds Musashi, but does not substantially bind other molecules in a sample. Examples of immunologically active portions of immunoglobulin molecules include F(ab) and F(ab′)2 fragments which can be generated by treating the antibody with an enzyme such as pepsin. The invention provides polyclonal and monoclonal antibodies that bind Musashi. The term “monoclonal antibody” or “monoclonal antibody composition”, as used herein, refers to a population of antibody molecules that contain only one species of an antigen binding site capable of immunoreacting with a particular epitope of Musashi. A monoclonal antibody composition thus typically displays a single binding affinity for a particular Musashi protein with which it immunoreacts.

Polyclonal anti-Musashi antibodies can be prepared as described above by immunizing a suitable subject with a Musashi immunogen. The anti-Musashi antibody titer in the immunized subject can be monitored over time by standard techniques, such as with an enzyme linked immunosorbent assay (ELISA) using immobilized Musashi. If desired, the antibody molecules directed against Musashi can be isolated from the mammal (e.g., from the blood) and further purified by well-known techniques, such as protein A chromatography to obtain the IgG fraction. At an appropriate time after immunization, e.g., when the anti-Musashi antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with a Musashi immunogen as described above, and the culture supernatants of the resulting hybridoma cells are screened to identify a hybridoma producing a monoclonal antibody that binds Musashi.

Any of the many well known protocols used for fusing lymphocytes and immortalized cell lines can be applied for the purpose of generating an anti-Musashi monoclonal antibody (see, e.g., Current Protocols in Immunology, supra; Galfre et al. (1977) Nature 266:55052; R.H. Kenneth, in Monoclonal Antibodies: A New Dimension In Biological Analyses, Plenum Publishing Corp., New York, N.Y. (1980); and Lemer (1981) Yale J. Biol. Med., 54:387-402). Moreover, the ordinarily skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Musashi, e.g., using a standard ELISA assay.

Additionally, recombinant anti-Musashi antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-Musashi antibody (e.g., monoclonal antibody) can be used to isolate Musashi by standard techniques, such as affinity chromatography or immunoprecipitation. An anti-Musashi antibody can facilitate the purification of natural Musashi from cells and of recombinantly produced Musashi expressed in host cells. Moreover, an anti-Musashi antibody can be used to detect Musashi protein (e.g., in a cellular lysate or cell supernatant) in order to evaluate the abundance and pattern of expression of the Musashi protein. Anti-Musashi antibodies can be used diagnostically to monitor protein levels in tissue as part of a clinical testing procedure, for example, to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials known in the art. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, .beta.-interferon, nerve growth factor, platelet derived growth factor, tissue plasminogen activator; or, biological response modifiers such as, for example, lymphokines, interleukin-1 (IL-1), interleukin-2 (IL-2), interleukin-6 (IL-6), granulocyte macrophase colony stimulating factor (GM-CSF), granulocyte colony stimulating factor (G-CSF), or other growth factors.

Techniques for conjugating such therapeutic moiety to antibodies are well known, see, e.g., Arnon et al., “Monoclonal Antibodies for Immunotargeting of Drugs in Cancer Therapy”, in Monoclonal Antibodies and Cancer Therapy, Reisfeld et al. (eds.), pp. 243-56 (Alan R. Liss, Inc. 1985); Hellstrom et al., “Antibodies for Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents in Cancer Therapy: A Review”, in Monoclonal Antibodies ‘84: Biological and Clinical Applications, Pinchera et al. (eds.), pp. 475-506 (1985); “Analysis, Results, and Future Prospective of The Therapeutic Use of Radiolabeled Antibody In Cancer Therapy”, in Monoclonal Antibodies for Cancer Detection and Therapy, Baldwin et al. (eds.), pp. 303-16 (Academic Press 1985), and Thorpe et al., “The Preparation and Cytotoxic Properties of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

In addition, antibodies of the invention, either conjugated or not conjugated to a therapeutic moiety, can be administered together or in combination with a therapeutic moiety such as a cytotoxin, a therapeutic agent or a radioactive metal ion. The order of administration of the antibody and therapeutic moiety can vary. For example, in some embodiments, the antibody is administered concurrently (through the same or different delivery devices, e.g., syringes) with the therapeutic moiety. Alternatively, the antibody can be administered separately and prior to the therapeutic moiety. Still alternatively, the therapeutic moiety is administered separately and prior to the antibody. In many embodiments, these administration regimens will be continued for days, months or years.

D. Pharmaceutical Compositions

The Musashi nucleic acid molecules, Musashi proteins, small molecules, and anti-Musashi antibodies (also referred to herein as “active compounds”) of the invention can be incorporated into pharmaceutical compositions suitable for administration. Such compositions typically comprise the nucleic acid molecule, protein, antibody, small molecules or combinations thereof and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity may, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight les than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled artisan. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. These can be prepared according to methods known to those skilled in the art, for example, as described in U.S. Pat. No. 4,522,811.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded.

The gene therapy vectors of the invention can be either viral or non-viral. Examples of plasmid-based, non-viral vectors are discussed in Huang et al. (1999) Nonviral Vectors for Gene Therapy (supra). A modified plasmid is one example of a non-viral gene delivery system. Peptides, proteins (including antibodies), and oligonucleotides may be stably conjugated to plasmid DNA by methods that do not interfere with the transcriptional activity of the plasmid (Zelphati et al. (2000) BioTechniques 28:304-315). The attachment of proteins and/or oligonucleotides may influence the delivery and trafficking of the plasmid and thus render it a more effective pharmaceutical composition.

### II. Methods

The nucleic acid molecules, proteins, protein homologues, and antibodies described herein can be used in one or more of the following methods: a) screening assays; b) detection assays, c) predictive medicine (e.g., diagnostic assays, prognostic assays, monitoring clinical trials, and pharmacogenomics); and d) methods of treatment (e.g., therapeutic and prophylactic). A Musashi protein regulates translation of cellular proteins and can thus be used for (i) regulation of cellular proliferation; and (ii) regulation of cellular differentiation. The isolated nucleic acid molecules of the invention can be used to express Musashi protein, to detect Musashi mRNA or a genetic lesion in a Musashi gene, and to modulate Musashi activity. In addition, the Musashi proteins can be used to screen drugs or compounds which modulate the Musashi activity or expression as well as to treat disorders characterized by insufficient or excessive production of Musashi protein or production of Musashi protein forms which have decreased or aberrant activity compared to Musashi wild type protein. In addition, the anti-Musashi antibodies of the invention can be used to detect and isolate Musashi proteins and modulate Musashi activity.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to Musashi proteins or biologically active portions thereof or have a stimulatory or inhibitory effect on, for example, Musashi expression or Musashi activity. Examples of biologically active portions of human Musashi include: amino acids about 21-100 encoding RNA recognition motif 1; amino acids about 110-189 encoding RNA recognition motif 2; amino acids about 88 -110 encoding a Nuclear localization sequence; and amino acids about 190-234 encoding a poly[A] binding domain (repression domain).

Among the screening assays provided by the invention are screening to identify molecules that prevent the RNA binding of Musashi. Screening assays can employ full-length Musashi or a portion of Musashi, such as the RNA binding domain, post-translational modification sites, or combinations thereof.

Screening assays can also be used to identify molecules that modulate a Musashi mediated increase in cell proliferation. For example, Musashi inhibits the translation of cell cycle inhibitor mRNA. Cell cycle progression can be measured using cell cycle markers known in the art.

In one embodiment, the invention provides assays for screening candidate or test compounds which bind to or modulate the activity of a Musashi proteins or polypeptides or biologically active portions thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

In one embodiment, an assay is one in which a polypeptide of the invention, or a biologically active portion thereof, is contacted with a test compound and the ability of the test compound to bind to the polypeptide determined. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

Determining the ability of the test compound to modulate the activity of Musashi or a biologically active portion thereof can be accomplished, for example, by determining the ability of the Musashi protein to bind to or interact with a Musashi target molecule. As used herein, a “target molecule” is a molecule with which a Musashi protein binds or interacts in nature. A Musashi target molecule can be a non-Musashi molecule or a Musashi protein or polypeptide of the present invention. Further, a Musashi target molecule may contain a Musashi binding motif, such as (G/A)UₙAGU (n=1-3) located in a hairpin structure. In one embodiment, a Musashi target molecule is mRNA of a protein involved in the regulation of the cell cycle, cell proliferation, cell differentiation, apoptosis, post-translational modification, or a combination thereof. Exemplary target molecules include mRNA transcripts of m-numb, CDKN1A, c-mos, cyclin 85, musashi, Dnmt1, p21WAF, Adenomatous Polyposis Coli, p27 and others. (See, de Sousa Abreu, R. et al. J. Biol. Chem. 2009 May 1; 284(18):12125-35, incorporated herein by reference).

Determining the ability of the test compound to modulate the activity of Musashi or a biologically active portion thereof can be accomplished, for example, by determining the ability of the Musashi protein to bind to or interact with any of the specific proteins listed in the previous paragraph as Musashi target molecules.

In yet another embodiment, an assay of the present invention is a cell-free assay comprising contacting a Musashi protein or biologically active portion thereof with a test compound and determining the ability of the test compound to bind to the Musashi protein or biologically active portion thereof. Binding of the test compound to the Musashi protein can be determined either directly or indirectly as described above. In one embodiment, a competitive binding assay includes contacting the Musashi protein or biologically active portion thereof with a compound known to bind Musashi to form an assay mixture, contacting the assay mixture with a test compound, and determining the ability of the test compound to interact with a Musashi protein, wherein determining the ability of the test compound to interact with a Musashi protein comprises determining the ability of the test compound to preferentially bind to Musashi or biologically active portion thereof as compared to the known binding compound.

In another embodiment, an assay is a cell-free assay comprising contacting Musashi protein or biologically active portion thereof with a test compound and determining the ability of the test compound to modulate (e.g., stimulate or inhibit) the activity of the Musashi protein or biologically active portion thereof. Determining the ability of the test compound to modulate the activity of Musashi can be accomplished, for example, by determining the ability of the Musashi protein to bind to or interact with a Musashi target molecule by one of the methods described above for determining direct binding. In an alternative embodiment, determining the ability of the test compound to modulate the activity of Musashi can be accomplished by determining the ability of the Musashi protein to further modulate a Musashi target molecule.

In another embodiment, modulators of Musashi expression are identified in a method in which a cell is contacted with a candidate compound and the expression of the Musashi promoter, mRNA or protein in the cell is determined. The level of expression of Musashi mRNA or protein in the presence of the candidate compound is compared to the level of expression of Musashi mRNA or protein in the absence of the candidate compound. The candidate compound can then be identified as a modulator of Musashi expression based on this comparison. For example, when expression of Musashi mRNA or protein is greater (statistically significantly greater) in the presence of the candidate compound than in its absence, the candidate compound is identified as a stimulator of Musashi mRNA or protein expression. Alternatively, when expression of Musashi mRNA or protein is less (statistically significantly less) in the presence of the candidate compound than in its absence, the candidate compound is identified as an inhibitor of Musashi mRNA or protein expression. The level of Musashi mRNA or protein expression in the cells can be determined by methods described herein for detecting Musashi mRNA or protein. The activity of the Musashi promoter can be assayed by linking the Musashi promoter to a reporter gene such as luciferase, secreted alkaline phosphatase, or beta-galactosidase and introducing the resulting construct into an appropriate vector, transfecting a host cell line, and measuring the activity of the reporter gene in response to test compounds.

This invention further pertains to novel agents identified by the above-described screening assays and uses thereof for treatments as described herein.

B. Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining Musashi protein and/or nucleic acid expression as well as Musashi activity, in the context of a biological sample (e.g., blood, serum, cells, tissue) to thereby determine whether an individual is afflicted with a disease or disorder, or is at risk of developing a disorder, associated with aberrant Musashi expression or activity. The invention also provides for prognostic (or predictive) assays for determining whether an individual is at risk of developing a disorder associated with Musashi protein, nucleic acid expression or activity. For example, mutations in a Musashi gene can be assayed in a biological sample. Such assays can be used for prognostic or predictive purpose to thereby prophylactically treat an individual prior to the onset of a disorder characterized by or associated with Musashi protein, nucleic acid expression or activity.

Another aspect of the invention provides methods for determining Musashi protein, nucleic acid expression or Musashi activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression or activity of Musashi in clinical trials.

These and other agents are described in further detail in the following sections.

1. Diagnostic Assays

An exemplary method for detecting the presence or absence of Musashi in a sample involves obtaining a sample from a test subject and contacting the sample with a compound or an agent capable of detecting Musashi protein or nucleic acid (e.g., mRNA, genomic DNA) that encodes Musashi protein such that the presence of Musashi is detected in the sample. An agent for detecting Musashi mRNA or genomic DNA is a labeled nucleic acid probe capable of hybridizing to Musashi mRNA or genomic DNA. The nucleic acid probe can be, for example, a full-length Musashi nucleic acid, such as the nucleic acid of SEQ ID NO:1-7 or a portion thereof, such as an oligonucleotide of at least 15, 30, 50, 100, 250, 500, 750 or more nucleotides in length and sufficient to specifically hybridize under stringent conditions to mRNA or genomic DNA. Other suitable probes for use in the diagnostic assays of the invention are described herein.

An agent for detecting Musashi protein can be an antibody capable of binding to Musashi protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The detection method of the invention can be used to detect Musashi mRNA, protein, or genomic DNA in a sample in vitro as well as in vivo. For example, in vitro techniques for detection of Musashi mRNA include Northern hybridizations and in situ hybridizations. In vitro techniques for detection of Musashi protein include enzyme linked immunosorbent assays (ELISAs), Western blots, immunoprecipitations and immunofluorescence. In vitro techniques for detection of Musashi genomic DNA include Southern hybridizations. Furthermore, in vivo techniques for detection of Musashi protein include introducing into a subject a labeled anti-Musashi antibody. For example, the antibody can be labeled with a radioactive marker whose presence and location in a subject can be detected by standard imaging techniques.

In one embodiment, the sample contains protein molecules from the test subject. Alternatively, the biological sample can contain mRNA molecules from the test subject or genomic DNA molecules from the test subject.

In another embodiment, the methods further involve obtaining a control sample from a control subject, contacting the control sample with a compound or agent capable of detecting Musashi protein, mRNA, or genomic DNA, such that the presence of Musashi protein, mRNA or genomic DNA is detected in the sample, and comparing the presence of Musashi protein, mRNA or genomic DNA in the control sample with the presence of Musashi protein, mRNA or genomic DNA in the test sample. In another embodiment, the compound or agent is capable of detecting post-translational status of Musashi. Preferably, the compound or agent is capable of detecting the phosphorylation status of Musahsi.

The invention also encompasses kits for detecting the presence of Musashi in a sample. The kit may comprise a labeled compound or agent capable of detecting Musashi protein or mRNA in a biological sample and means for determining the amount of Musashi in the sample.

For antibody-based kits, the kit may comprise, for example: (1) a first antibody (e.g., attached to a solid support) which binds to Musashi protein; and, optionally, (2) a second, different antibody which binds to Musashi protein or the first antibody and is conjugated to a detectable agent. For oligonucleotide-based kits, the kit may comprise, for example: (1) a oligonucleotide, (e.g., a detectably labeled oligonucleotide), which hybridizes to a Musashi nucleic acid sequence or (2) a pair of primers useful for amplifying a Musashi nucleic acid molecule.

The kit may also comprise, a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for use.

2. Prognostic Assays

The methods described herein can furthermore be utilized as diagnostic or prognostic assays to identify subjects having or at risk of developing a disease or disorder associated with aberrant Musashi expression or activity. For example, the assays described herein, such as the preceding diagnostic assays or the following assays, may be utilized to identify a subject having or at risk of developing a disorder associated with Musashi protein, nucleic acid expression or activity. Alternatively, the prognostic assays may be utilized to identify a subject having or at risk for developing such a disease or disorder. Thus, the present invention provides a method in which a test sample is obtained from a subject and Musashi protein or nucleic acid (e.g., mRNA, genomic DNA) is detected, wherein the presence of Musashi protein or nucleic acid is diagnostic for a subject having or at risk of developing a disease or disorder associated with aberrant Musashi expression or activity. As used herein, a “test sample” refers to a biological sample obtained from a subject of interest. For example, a test sample can be a biological fluid (e.g., serum), cell sample, or tissue. Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant Musashi expression or activity. Exemplary diseases include, without limitation, cancer, Alzheimer's disease, Pick disease, Parkinson disease, Barrett's esophagus, esophageal adenocarninoma, aberrant cell proliferation associated diseases, and aberrant cell differentiation associated diseases.

The methods of the invention can also be used to detect genetic lesions or mutations in a Musashi gene, thereby determining if a subject with the lesioned gene is at risk for a disorder characterized by aberrant cell proliferation and/or differentiation. In preferred embodiments, the methods include detecting, in a sample from the subject, the presence or absence of a genetic lesion characterized by at least one of an alteration affecting the integrity of a gene encoding a Musashi-protein, or the mis-expression of the Musashi gene. For example, such genetic lesions can be detected by ascertaining the existence of at least one of 1) a deletion of one or more nucleotides from a Musashi gene; 2) an addition of one or more nucleotides to a Musashi gene; 3) a substitution of one or more nucleotides of a Musashi gene; 4) a chromosomal rearrangement of a Musashi gene; 5) an alteration in the level of a messenger RNA transcript of a Musashi gene; 6) aberrant modification of a Musashi gene, such as of the methylation pattern of the genomic DNA; 7) the presence of a non-wild type splicing pattern of a messenger RNA transcript of a Musashi gene (e.g., caused by a mutation in a splice donor or splice acceptor site); 8) a non-wild type level of a Musashi-protein; 9) allelic loss of a Musashi gene; and 10) inappropriate post-translational modification of a Musashi-protein. As described herein, there are a large number of assay techniques known in the art which can be used for detecting lesions in a Musashi gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the Musashi gene (see, e.g., Abravaya et al. (1995) Nucleic Acids Res. 23:675-682). This method can include the steps of collecting a sample from a patient, isolating nucleic acid (e.g., genomic, mRNA or both) from the sample, contacting the nucleic acid sample with one or more primers which specifically hybridize to a Musashi gene under conditions such that hybridization and amplification of the Musashi-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In other embodiments, genetic mutations in Musashi can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in Musashi can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the Musashi gene and detect mutations by comparing the sequence of the sample Musashi with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the Musashi gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type Musashi sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with 51 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al (1988) Proc. Natl Acad Sci USA 85:4397; Saleeba et al (1992) Methods Enzymol. 217:286-295. In an embodiment, the control DNA or RNA can be labeled for detection.

In other embodiments, alterations in electrophoretic mobility will be used to identify mutations in Musashi genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet Anal Tech Appl 9:73-79). Single-stranded DNA fragments of sample and control Musashi nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In an embodiment, the subject method utilizes heteroduplex analysis to separate double stranded heteroduplex molecules on the basis of changes in electrophoretic mobility (Keen et al. (1991) Trends Genet 7:5).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a Musashi gene.

3. Monitoring of Effects during Therapeutic Treatment

Monitoring the influence of agents (e.g., drugs, compounds) on the expression or activity of Musashi (e.g., the ability to modulate aberrant cell proliferation and/or differentiation) can be applied not only in basic drug screening, but also in therapeutic treatments. For example, the effectiveness of an agent determined by a screening assay as described herein to increase Musashi gene expression, protein levels, or upregulate Musashi activity, can be monitored in subjects exhibiting decreased Musashi gene expression, protein levels, or downregulated Musashi activity. Alternatively, the effectiveness of an agent determined by a screening assay to decrease Musashi gene expression, protein levels, or downregulated Musashi activity, can be monitored in clinical trials of subjects exhibiting increased Musashi gene expression, protein levels, or upregulated Musashi activity. In such clinical trials, the expression or activity of Musashi and, preferably, other genes that have been implicated in, for example, a cellular proliferation disorder can be used as a “read out” or markers of the immune responsiveness of a particular cell.

For example, and not by way of limitation, genes, including Musashi, that are modulated in cells by treatment with an agent (e.g., compound, drug or small molecule) which modulates Musashi activity (e.g., identified in a screening assay as described herein) can be identified. Thus, to study the effect of agents on cellular proliferation disorders, for example, in a clinical trial, cells can be isolated and RNA prepared and analyzed for the levels of expression of Musashi and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of Musashi or other genes. In this way, the gene expression pattern can serve as a marker, indicative of the physiological response of the cells to the agent. Accordingly, this response state may be determined before, and at various points during, treatment of the individual with the agent.

In an embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a Musashi protein, mRNA, or genomic DNA in the preadministration sample; (iii) obtaining one or more post-administration samples from the subject; (iv) detecting the level of expression or activity of the Musashi protein, mRNA, or genomic DNA in the post-administration samples; (v) comparing the level of expression or activity of the Musashi protein, mRNA, or genomic DNA in the pre-administration sample with the Musashi protein, mRNA, or genomic DNA in the post administration sample or samples; and (vi) altering the administration of the agent to the subject accordingly. For example, increased administration of the agent may be desirable to increase the expression or activity of Musashi to higher levels than detected, i.e., to increase the effectiveness of the agent. Alternatively, decreased administration of the agent may be desirable to decrease expression or activity of Musashi to lower levels than detected, i.e., to decrease the effectiveness of the agent.

4. Transcriptional Profiling

The Musashi nucleic acid molecules described herein, including small oligonucleotides, can be used in transcriptionally profiling. For example, these nucleic acids can be used to examine the expression of Musashi in normal tissue or cells and in tissue or cells subject to a disease state, e.g., tissue or cells derived from a patient having a disease of interest or cultured cells which model or reflect a disease state of interest, e.g., cells of a cultured tumor cell line. By measuring expression of Musashi, together or individually, a profile of expression in normal and disease states can be developed. This profile can be used diagnostically and to examine the effectiveness of a therapeutic regime.

C. Methods of Treatment

The present invention provides for both prophylactic and therapeutic methods of treating a subject at risk of (or susceptible to) a disorder or having a disorder associated with aberrant Musashi expression or activity, examples of which are provided herein.

1. Prophylactic Methods

In one aspect, the invention provides a method for preventing in a subject, a disease or condition associated with an aberrant Musashi expression or activity, by administering to the subject an agent which modulates Musashi expression or at least one Musashi activity. Subjects at risk for a disease which is caused or contributed to by aberrant Musashi expression or activity can be identified by, for example, any or a combination of diagnostic or prognostic assays as described herein. Administration of a prophylactic agent can occur prior to the manifestation of symptoms characteristic of the Musashi aberrancy, such that a disease or disorder is prevented or, alternatively, delayed in its progression. Depending on the type of Musashi aberrancy, for example, a Musashi agonist or Musashi antagonist agent can be used for treating the subject. The appropriate agent can be determined based on screening assays described herein.

2. Therapeutic Methods

Another aspect of the invention pertains to methods of modulating Musashi expression or activity for therapeutic purposes. The modulatory method of the invention involves contacting a cell with an agent that modulates one or more of the activities of Musashi protein activity associated with the cell. An agent that modulates Musashi protein activity can be an agent as described herein, such as a nucleic acid or a protein, a naturally-occurring cognate ligand of a Musashi protein, a peptide, a Musashi peptidomimetic, or other small molecule. In one embodiment, the agent stimulates one or more of the biological activities of Musashi protein. Examples of such stimulatory agents include active Musashi protein and a nucleic acid molecule encoding Musashi that has been introduced into the cell. In another embodiment, the agent inhibits one or more of the biological activities of Musashi protein. Examples of such inhibitory agents include antisense Musashi nucleic acid molecules and anti-Musashi antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a Musashi protein or nucleic acid molecule or a disorder related to Musashi expression or activity. In one embodiment, the method involves administering an agent (e.g., an agent identified by a screening assay described herein), or combination of agents that modulates (e.g., upregulates or downregulates) Musashi expression or activity. In another embodiment, the method involves administering a Musashi protein or nucleic acid molecule as therapy to compensate for reduced or aberrant Musashi expression or activity. Stimulation of Musashi activity is desirable in situations in which Musashi is abnormally downregulated and/or in which increased Musashi activity is likely to have a beneficial effect. Conversely, inhibition of Musashi activity is desirable in situations in which Musashi is abnormally upregulated, e.g., in myocardial infarction, and/or in which decreased Musashi activity is likely to have a beneficial effect.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A, non-limiting example of stringent hybridization conditions are hybridization in 6× sodium chloride/sodium citrate (SSC) at about 45° C., followed by one or more washes in 0.2.×SSC, 0.1% SDS at 50-65° C. (e.g., 50° C. or 60° C. or 65° C.) Preferably, the isolated nucleic acid molecule of the invention that hybridizes under stringent conditions corresponds to a naturally-occurring nucleic acid molecule. As used herein, a “naturally-occurring” nucleic acid molecule refers to an RNA or DNA molecule having a nucleotide sequence that occurs in a human cell in nature (e.g., encodes a natural protein).

As used herein, the terms “gene” and “recombinant gene” refer to nucleic acid molecules comprising an open reading frame encoding a Musashi protein, preferably a mammalian Musashi protein.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences (preferably protein encoding sequences) that which naturally flank the nucleic acid (i.e., sequences located at the 5′ and 3′ ends of the nucleic acid) in the genomic DNA of the organism from which the nucleic acid is derived. For example, in various embodiments, the isolated Musashi nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

Musashi is a member of a family of molecules (the Musashi family) having certain conserved structural and functional features. The term “family” when referring to the protein and nucleic acid molecules of the invention is intended to mean two or more proteins or nucleic acid molecules having a common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

As used interchangeably herein a “Musashi activity”, “biological activity of Musashi” or “functional activity of Musashi” refers to an activity exerted by a Musashi protein, polypeptide or nucleic acid molecule on a Musashi responsive cell, target mRNA, or target protein as determined in vivo or in vitro, according to standard techniques. Musashi may act as a cell proliferation or cell differentiation regulator. A Musashi activity can be a direct activity such as an association with a second protein or mRNA. A Musashi activity may be an indirect activity such as a cellular signaling activity mediated by interaction of the Musashi protein with a second protein or mRNA.

The term “sample” refers to a cell, a population of cells, biological samples, and subjects, such as mammalian subjects. The term “biological sample” refers to tissues, cells and biological fluids isolated from a subject, as well as tissues, cells and fluids present within a subject.

The term “post-translational modification” refers to modifications made to a protein after the protein amino acid sequence has been translated from the mRNA sequence. Exemplary post-translational modifications include phosphorylation, myristoylation, palmitoylation, isoprenylation, prenylation, farnesylation, geranylgeranylation, glypiation, lipoylation, acylation, and alkylation.

The term “Musashi agent” refers to any molecule capable of modulating Musashi activity. Exemplary Musashi agents include, without limitation, a compound, drug, small molecule, peptide, oligonucleotide, protein, antibody, and combinations thereof. Musashi agents may be synthetic or naturally occurring. A Musashi agent may be a molecule identified in a screening assay as described herein.

The term “Musashi indicator” refers to any molecule capable of detecting the post-translational status of Musashi protein. In one embodiment, the Musashi indicator is capable of detecting the phosphorylation status of Musashi protein. A suitable Musashi indicator may be a compound, drug, small molecule, peptide, oligonucleotide, protein, antibody, and combinations thereof.

The term “Musashi associated disease” refers to any disease or condition caused by or exhibiting aberrant Musashi expression or activity. This term includes any disease associated with aberrant cell proliferation, cell differentiation, apoptosis, protein modification or combinations thereof. Exemplary conditions and diseases include, without limitation, cancer, neurodegenerative disease, and other diseases known in the art associated with aberrant cell proliferation, cell differentiation, apoptosis, or protein modification.

### EXAMPLES

The following examples are simply intended to further illustrate and explain the present invention. The invention, therefore, should not be limited to any of the details in these examples.

### Example 1

### Materials and Methods for the Identification and Characterization of Musashi Phosphorylation and Regulation

The following methods and materials were used in the Examples herein provided to further illustrate and explain the present invention.

Plasmid Construction. The plasmids encoding GST Xenopus Musashi1 (xMsi1), GST-mMsi1, GST-Wee107 and GST-vRaf were constructed using molecular cloning techniques. For example, the pGEM Ringo construct was generated by designing PCR primers to amplify the full length Ringo sequence with a 5′ Cla I site and a 3′ Xho I site. cDNA was made from RNA from immature Xenopus oocytes using the reverse PCR primer and Superscript III. Full length Ringo was amplified using Platinum Pfx and the PCR product digested with Cla I and Xho I and ligated into Cla I/Xho I digested pXen1. The GST was excised from pXen Ringo with Ncol and Cla I, blunted with Mung Bean Nuclease and the remaining plasmid re-ligated to generate pGEM Ringo. Also by example, the pGEMFIuc construct or the firefly luciferase vector pGEM_luc2 was constructed by cloning the luciferase 2 gene from pLuc2-IRES2-Ds-Red-Express vector into the pGEM-4Z vector between a 5′ Ncol site and a 3′ SalI site. Also, the pGEMFluc xMsi1 wt 3′ UTR reporter construct was generated by designing PCR primers to amplify the last 100 nucleotides of NRP-1 B (Xenopus Musashi1) mRNA 3′ UTR, with a 5′ SacI site (5′-CGGAGCTCCAATACTGCAATGTACAATGTACTGC -3′) and a 3′ BamHI site (5′-GCGGGATCCTGAATAAAATTCAATTTATTTTG-3′). cDNA was prepared using RNA extracted from immature Xenopus oocytes using the reverse PCR primer and Superscript III. The 100-nucleotide portion of NRP-1 B 3′ UTR was amplified using Platinum Taq and the PCR product digested with SacI and BamHI and ligated into SacI/BamHI-digested pGEM_luc2.

The following additional constructs were made by altering the above described constructs. pXen1 GST xMsi1 S322A—The serine 322 of the wild type xMsi1 sequence was replaced to an alanine by site-directed mutagenesis of the pXen1 GST xMsi1 wild-type plasmid. pXen1 GST xMsi1 S322E—The serine 322 of the wild type xMsi1 sequence was replaced to a glutamic acid by site-directed mutagenesis of the pXen1 GST xMsi1 wild-type plasmid. pGEM Ringo D83A—The aspartic acid at position 83 of the wild type Ringo sequence was replaced with alanine by site-directed mutagenesis of the pGEM Ringo plasmid. pGEMFIuc xMsi1 mbm 3′ UTR reporter—The Musashi-binding mutant of NRP-1 B UTR (AUAGU ->AUccU) was made by site-directed mutagenesis of the pGEMFIuc xMsi1 wt 3′ UTR reporter. pGEMGST xMsi1 wt 3′ UTR reporter—The NRP-1 B wt UTR was cloned by cutting it from pGEMFIuc xMsi1 3′ UTR construct at the 5′ EcoRI site and a 3′ BamHI site, blunted with Klenow and ligated into a Xbal digested Klenow blunted pGEM GST vector. pGEMGST xMsi1 mbm 3′ UTR reporter—The musashi-binding mutant of NRP-1 B 3′ UTR was cloned by cutting it from pGEMFluc xMsi1 mbm 3′ UTR construct at the 5′ EcoRI site and a 3′ BamHI site, blunted with Klenow and ligated into a Xbal digested Klenow blunted pGEM GST vector.

EMSA Competition constructs—For EMSA competition assays the pGEMFIuc NRP-1 B wt UTR and Musashi-binding mutant UTR constructs were digested with EcoRI to excise the luciferase gene, re-ligated and used to generate unlabelled competitor RNA.

All constructs were transcribed with SP6 RNA Polymerase and injected at 23 ng/oocyte.

RNA Electrophoretic Mobility Shift Assays. GST fusion proteins were in vitro transcribed/translated using TNT SP6-coupled Reticulocyte Lysate System (Promega). 5′ biotin-labeled RNA oligonucleotide probes were synthesized by Integrated DNA Technologies. Unlabeled competitor mRNAs were transcribed in vitro. An 80 fmol portion of labeled probe was incubated with 1 μl of reticulocyte lysate and 200 molar excess of unlabeled mRNA in binding buffer (50 mM Tris pH 7.5, 20 mM KCI, 150 mM NaCI, 2 mM EGTA, 0.05% NP-40, 6 mM DTT, 8 U RNaseOUT) in a final volume of 20 μl. The binding reaction was incubated at room temperature for 20 min and then 1 μl of 20 mg/ml heparin was added and incubated for a further 20 min. A 5 μl volume of the binding reaction was run on a 6% DNA retardation gel and transferred to Biodyne B membranes. Biotinylated RNA was detected using Chemiluminescent Nucleic Acid Detection Module, with the modification that incubation with the streptavidin-HRP conjugate was for 40 min. Image collection was performed using an Alphalnnotech Chemilmager.

Semi-quantitative PCR. cDNAs for semi-quantitative PCR analysis were synthesized using RNA ligation-coupled RT-PCR. For the PCR reaction, 2 μl of cDNA was used with 3 mM MgCl₂, 1 mM firefly luciferase primers (forward-5′-GAGTACTTCGAGATGAGC-3′, reverse-5′-CACGA AGTCGTACTCGTT-3′) and 0.25 mM cyclin B1 primers (forward-5′-GGCTTGAGACCTCGTACAGC-3′, reverse-5′-CAGGGAGGCAACCAGATG-3′) for the indicated number of cycles.

Oocyte culture and microinjections. Xenopus oocytes were isolated and cultured. Oocytes were induced to mature with 2 μg/ml progesterone. The rate of germinal vesicle breakdown (GVBD) was scored morphologically by observing the appearance of a white spot on the animal pole. Because oocytes from different frogs mature at different rates in response to progesterone, the culture times were standardized between experiments to the time taken for 50% of oocytes to undergo GVBD. Where indicated, oocytes were pre-treated with 50 μM MEK inhibitor, UO126 or DMSO vehicle for 30 minutes.

Luciferase reporter assays. Oocytes were injected with 0.1 ng of firefly luciferase mRNA and 0.35 pg of Renilla luciferase control mRNA, and incubated at 18° C. for 18 h. Three pools of 5 oocytes were lysed for each experimental point and lysed in 50 μl of Passive lysis buffer. A 10 μl portion of lysate was analyzed for Renilla and firefly luciferase activity using the Dual-Luciferase Assay System on a TD-20/20 Turner Designs luminometer. Mean values and standard deviation were determined for each experimental point, with the ratio of firefly to Renilla luciferase normalized to 1 the β-globin reporter construct (which was arbitrarily set to 1.0).

Mass Spectrometry. Oocytes were microinjected RNA encoding a GST tagged form of Xenopus Musashi1 and following an 18 hour culture period to allow expression of the introduced Musashi protein, oocytes were split into two pools and either left untreated (time matched control) or stimulated with progesterone (treated). Oocyte lysate was prepared when progesterone oocytes had fully matured. The ectopic Musashi1 protein was recovered over glutathione Sepharose beads, resolved by SDS-PAGE, and visualized by Coomassie-staining (FIG. 2C). Under these conditions, the GST-Musashi1 protein appeared to undergo a progesterone-dependent mobility shift. Control and treated GST-Musashi1 protein bands were excised, in-gel digested with 100 ng of GIuC, and prepared for MALDI mass spectrometric analysis. MALDI mass spectra were collected with a PerkinElmerSciex prOTOF, while MS² and MS³ spectra were collected with a Thermo vMALDI-LTQ. Phosphorylation of the GST-Musashil fusion protein was mapped to Ser524 on peptide 514-549 by monitoring for a neutral loss of 98Da in MS² and sequencing of the neutral loss ion in MS³. This site corresponds to S322 of the native Musashi1 protein.

Polyadenylation assays. cDNAs for polyadenylation assays were synthesized using RNA ligation-coupled PCR. The increase in PCR product length is specifically due to extension of the poly[A] tail. The primer used to analyze endogenous Xenopus Musashi1 mRNA polyadenylation was 5′-CAATACTGCAATGTACAATGTACTGC-3′.

Antisense oligodeoxynucleotide injections. Antisense oligodeoxynucleotides targeting endogenous Musashi1 and Musashi2 mRNAs, Ringo mRNA or a randomized control oligonucleotide sequence 5′-TAGAGAAGATAATCGTCATCTTA-3′. Oocytes were incubated at 18° C. for 16 hours followed by injection of mRNA encoding GST rescue proteins with or without progesterone treatment as indicated.

Cell culture. Primary neural stem/progenitor cells were cultured from embryonic rat hippocampal/cortical tissue and grown as neurospheres in low adhesion dishes in serum-free medium supplemented with EGF and bFGF. Differentiation was induced by plating enzymatically and mechanically dispersed cells on tissue culture-treated dishes in the absence of bFGF and EGF and in the presence of retinoic acid. The SH-SY5Y human neuroblastoma cell line (ATCC) was cultured for growth and differentiation under the same conditions as the primary NSPCs.

Antibodies. Antisera for phosphorylation specific Musashi1 S322 were generated by immunizing rabbits with the peptide VSSYISAAS(phospho)PAPSTGF. The antibodies were affinity purified through a peptide-affinity column and used at 1:1000. Abcam antibodies to Musashi1 were used at 1:1000. Sigma Tubulin antibodies were used at 1:20,000. The phospho-specific Cdc2 antibody was used at 1:1000 and detects the inhibitory Tyr15 phosphorylation. The phospho-specific MAP kinase antibody (Cell Signaling) was used at 1:1000 and detects the activating phosphorylations at Thr202/Tyr204. The GST antibody (Santa Cruz) was used at 1:1000. The Xenopus Ringo antibody was used at 1:1000.

Western blotting. Oocytes were lysed in NP40 lysis buffer containing sodium vanadate and a protease inhibitor cocktail. Where indicated, a portion of the lysate was transferred to STAT-60 for RNA extraction. Protein lysates were then spun, clarified and transferred immediately to 1× sample buffer. The lysates were run on a 10% Nupage gel and transferred to a 0.2 pm-pore-size nitrocellulose filter. The membrane was blocked with 1% bovine serum albumin in TBST for 60 min at room temperature. Following incubation with primary antibody, filters were incubated with horseradish peroxidase conjugated secondary antibody using enhanced chemiluminescence in a Fluorchem 8000 Advanced Imager.

Statistical analyses. All quantitated data are presented as mean+/−SEM. Statistical significance was assessed by one-way analysis of variance (ANOVA) followed by the Bonferroni post hoc test or by Student's t-test when only two groups were compared. A probability of p <0.05 was adopted for statistical significance.

### Example 2

### Regulation of Musashi mRNA Translation

The Musashi RNA-binding protein promotes physiological stem cell self-renewal and pathological tumor growth by repressing inhibitors of cell cycle progression at the level of mRNA translation. During differentiation of stem cells, inhibition of Musashi target mRNA translation is reversed. During Xenopus oocyte maturation, progesterone-stimulated polyadenylation and translational activation of select mRNAs occurs in a distinct temporal pattern and can be classed as “early” (prior to germinal vesicle(nuclear) breakdown (“GVBD”), e.g., Mos) or “late” (coincident with, or after, gVBD, e.g., cyclin B1). Musashi mediates the activation of early mRNA targets as down-regulation of Musashi function through antisense oligonucleotide (as) treatment inhibits both progesterone-stimulated early class mRNA translation and Xenopus oocyte maturation.

To determine if Musashi is regulated by progesterone, Musashi levels were detected in progesterone stimulated oocytes using methods described in Example 1. Musashi protein levels were observed to increase in response to progesterone stimulation (FIG. 1A). Lysates from oocytes treated with or without progesterone were analyzed by western blot for endogenous Musashi1 protein accumulation at GVBD₅₀. Quantifications of fold changes in Musashi1 levels (as indicated below western) were normalized to tubulin from the same sample, and levels in immature oocytes (Imm) arbitrarily set to 1.0. The bar graph of FIG. 1B represents data from 3 independent experiments with S.E.M. (Student's t-test; p<0.01).

Musashi mRNA was polyadenylated early in response to progesterone stimulation (FIG. 1C). Oocytes treated with or without progesterone for the indicated times were analyzed for endogenous Musashi1 mRNA polyadenylation by RNA ligation-coupled PCR. An increase in size of the PCR products is indicative of polyadenylation. Polyadenylation of the late class cyclin A1 mRNA in the same samples is shown by comparison and occurs after completion of GVBD.

Examination of Musashi1 mRNA revealed the presence of a consensus Musashi Binding Element (MBE) within the 3′ untranslated region (UTR). The MBE within the Musashi mRNA 3′UTR directs translational control by Musashi (FIGS. 1D, 1E, and 1F). To confirm that the MBE within the Musashi1 mRNA 3′ UTR interacted with Xenopus Musashi protein, an RNA EMSA was conducted using the Musashi1 3′ UTR in competition with a biotinylated Mos 3′ UTR probe which contains a functional MBE. A Musashi1 3′ UTR with a disrupted MBE or the wild-type Mos 3′ UTR served as negative and positive controls, respectively. A schematic showing the position of the polyadenylation hexanucleotide (hexagon), MBE (square) and CPE (shaded circle) are shown for each construct (FIG. 1 D). The disrupted mutant MBE (AUAGU→AUccU) is shown as an “X”. The N-terminal mRNA binding domain of Musashi1 (N-Msi) or an RNA binding mutant form (N-Msi bm) were expressed as GST fusion proteins in rabbit reticulocyte lysates for use in the EMSA reactions (FIG. 1 E). RNA EMSA using the indicated unlabeled RNA probes to compete for Mos 3′ UTR interaction with Musashil was conducted. The Mos 3′ UTR MBE binds specifically to the N-terminus of Musashil but not to an RNA-binding mutant of this protein (FIG. 1 F). The Musashi1 3′ UTR, like the Mos 3′ UTR, efficiently competed with the biotinylated Mos 3′ UTR probe to prevent formation of a specific complex with N-Msi. Thus, Musashi1 protein binds specifically to the MBE in the Musashi1 mRNA 3′ UTR. Several non-specific complexes, detected with unprogrammed reticulocyte lysate are indicated by open arrowheads. A representative experiment is shown. An MBE is also present in the same position within Xenopus tropicalis Musashi1 3′ UTR (Accession NM_{—}001011470), suggesting a conservation of Musashi1 auto-regulatory potential in amphibia. The Xenopus Musashi1 mRNA 3′ UTR also contains a CPE, located 3′ of the polyadenylation hexanucleotide. The CPE may function to maintain translation of Musashi1 mRNA in fully mature oocytes.

The Musashi1 3′ UTR was linked 3′ of an RNA encoding the GST epitope and injected into immature oocytes. Oocytes were stimulated with progesterone for 6 hours to mature the oocytes, total RNA was prepared and the polyadenylation of the injected reporter mRNA assessed by RNA ligation-coupled PCR using a primer homologous to the GST coding region (FIG. 1G). The wild-type xMsi1 3′UTR was polyadenylated upon progesterone stimulation (retarded mobility of the PCR product relative to that present in immature oocytes as seen above the dashed reference line). Polyadenylation directed by the Musashi1 3′ UTR was completely abrogated when the MBE was disrupted (Msi mbm). No progesterone-dependent polyadenylation of a control reporter under the control of the unregulated β-globin 3′ UTR was observed. Polyadenylation of endogenous late class Wee1 mRNA in the same samples was used as an internal control.

Translation of a reporter linked to the Musashi 3′ UTR was dependent on the MBE. Schematics representing the 3′ UTR firefly luciferase reporter constructs utilized with consensus Musashi binding element (MBE, black square), consensus cytoplasmic polyadenylation element (CPE, white circle) and the consensus polyadenylation hexanucleotide (gray hexagon) are shown in FIG. 1H). The disrupted MBE in the Musashi binding mutant (mbm) UTR is shown as an “X”. Oocytes were injected with RNA encoding Renilla luciferase and the indicated Musashi1 (Msi1) 3′ UTR firefly luciferase reporter constructs and incubated for 16 hours. After progesterone treatment, the oocytes were lysed at GVBD and analyzed for both Renilla and Firefly luciferase activities (FIG. 1I). The plot shows an average ratio of firefly luciferase activity for the Musashi1 reporters relative to co-injected Renilla luciferase from three independent experiments. All ratios were normalized to that seen with the firefly luciferase fused to the unregulated control 13 globin 3′ UTR in the absence of progesterone (arbitrarily set to 1.0). Error bars indicate the SEM and differences were significant as assessed by a Bonferroni test (* P<0.01). The levels of the reporters were equivalent, indicating that the difference in luciferase activity is a consequence of differential reporter translation (FIG. 1J). Semi-quantitative PCR on RNA isolated from the same samples used to analyze Firefly luciferase expression was conducted (FIG. 1I). Firefly reporter RNA and endogenous cyclin B1 were PCR amplified for different cycle numbers as indicated and visualized after separation through a 2% agarose gel. No significant differences in stability of the different constructs were detected with or without progesterone treatment.

Translational activation of the endogenous Musashi1 mRNA was mediated by Musashi. Musashi function was attenuated in immature oocytes with antisense oligonucleotides targeting both endogenous Musashi1 and Musashi2 mRNAs (Msi AS, FIG. 1K; UI, uninjected, no antisense injection. Con AS, control antisense injection. Msi AS, Musashi antisense, no rescue. I, immature oocytes, no progesterone). Oocytes were subsequently re-injected with RNA encoding a GST tagged form of the wild-type Musashi1 (Msi AS+GST Msi WT) and stimulated with progesterone. When 50% of the population matured, oocytes were segregated into those that had not (−) or had (+) completed GVBD and total RNA was isolated and analyzed for endogenous Musashi1 mRNA polyadenylation by RNA ligation-coupled PCR. An increase in PCR product size above that seen in immature oocytes (Imm) is indicative of polyadenylation (dotted reference line). Time-matched samples were also prepared from progesterone-stimulated Msi AS oocytes (no rescue) which failed to mature and immature oocytes.

Polyadenylation-dependent translational activation of the Musashi mRNA was attenuated in oocytes treated with Musashi antisense oligonucleotides and was rescued through ectopic expression of Musashi protein. The timing and dependence upon Musashi function for translational control of endogenous Musashi1 mRNA indicates that translational activation of the Musashi mRNA is mediated through activation of Musashi protein.

### Example 3

### Musashi1 Undergoes Phosphorylation at a Conserved Serine

Musashi is required to mediate oocyte maturation (FIG. 2A). Musashi function was attenuated by injection of antisense DNA oligonucleotides targeting the endogenous Musashi1 and Musashi2 mRNAs. Oocytes were re-injected with water (no rescue) or RNA encoding GST-tagged wild-type Musashi1 and either left untreated (−prog) or stimulated with progesterone (+prog). The ability of the ectopic Musashi to rescue cell cycle progression was assessed (% GVBD). Results from two independent experiments are shown. A GST western blot confirmed expression of the ectopic Musashi1 protein (arrowhead, FIG. 2B). The over-expression of Musashi is not sufficient to induce maturation in the absence of progesterone stimulation. This indicates that Musashi target mRNAs are not translated per se, but require an activation process.

To determine if Musashi is subject to activating post-translational modification, tandem mass spectrometry was utilized to compare Musashi protein isolated from immature and from progesterone-stimulated oocytes. A unique site of phosphorylation in progesterone-treated samples was mapped to serine 322 (S322) of xenopus Musashi 1 protein (FIGS. 2C, 2D, and 2E). The sequence flanking the identified serine phosphorylation site (FIG. 2F, arrowhead) is conserved in human (hu), mouse (mu), Xenopus (xe) and drosophila (dr) Musashi1 and Musashi2 isoforms.

Musashi is phosphorylated on S322 in response to progesterone stimulation (FIG. 2D). Musashi (WT) or a non-phosphorylatable mutant Musashi (S332A) were expressed in oocytes as GST-fusion proteins and then stimulated with progesterone. Protein and RNA samples were prepared at the indicated time points. At 5.5 hours, 50% of the injected oocytes had completed GVBD and they were segregated based on whether they had (+) or had not (−) completed GVBD. A western blot of oocyte lysates with antibody specific to the S322 phosphorylated form of Musashil demonstrates progesterone-stimulated phosphorylation that is not observed in the S322A mutant Musashi (FIG. 2G, upper panel). A GST western blot shows equivalent levels of the expressed proteins (FIG. 2G, lower panel).

The Musashi mRNA target, Mos, is activated coincident with Musashil S322 phosphorylation (FIG. 2H). Total RNA from samples shown in FIG. 2G was analyzed for polyadenylation of the endogenous Mos mRNA. Increased PCR product size indicates polyadenylation and this initiates approximately 3 hours after progesterone stimulation (FIG. 2H, asterisk). Endogenous Musashi1 was phosphorylated on S322 in response to progesterone stimulation (FIG. 21). Uninjected oocytes were stimulated with progesterone and western blots of protein lysate prepared at the indicated time points were assayed with antisera specific for S322 phosphorylated Musashi1 . Quantification of maximum progesterone-induced changes in phospho-Musashi1 levels normalized to tubulin from the same samples, revealed a 1.25+/−0.11 fold increase (Student's t-test; p<0.05, n=4) relative to levels in immature oocytes (Imm).

Mammalian Musashi1 was phosphorylated on S337 in response to progesterone stimulation (FIG. 2J). Immature Xenopus oocytes were injected with RNA encoding GST fused to murine Musashi1 (mMsi1) and were stimulated with progesterone (prog) or left untreated (Imm). When 50% of the progesterone-stimulated oocyte population completed GVBD, oocytes were segregated as described in (C) and analyzed by western blot with phospho S322 Musashi-specific antiserum and with GST antiserum to show levels of the expressed protein (GST mMsi1). The appearance of S337 phosphorylated mammalian Musashi1 is coincident with a gel mobility shift of the expressed Musashi protein.

Musashi1 was phosphorylated on S337 in differentiating embryonic rat neural stem/progenitor cells (FIG. 2K). Cell lysate was prepared from proliferating (Pro) neural stem/progenitor cells or after 1 hour of exposure to differentiation conditions (Diff) and total Musashi1 and phospho-5337 Musashi1 were detected by western blot with appropriate antisera. Musashi1 was also phosphorylated on S337 in differentiating SH-SY5Y neuroblastoma cells (FIG. 2L). Cell lysate was prepared from proliferating (Pro) human SH-SY5Y neuroblastoma cells or from SH-SY5Y cells induced to differentiate for the indicated times and phospho-S337 Musashi1 detected by western blot. In this experiment, GAPDH protein levels serve as an internal control for protein loading. These findings temporally position this modification to mediate activation of Musashi translational control function.

### Example 4

### Musashi1 S322 Phosphorylation Facilitates Oocyte Maturation and Target mRNA Translational Activation

Inhibition of Musashi1 S322 phosphorylation attenuates oocyte maturation (FIGS. 3A and 3B). Oocytes were injected with antisense oligonucleotides to ablate endogenous Musashi function and subsequently reinjected with water (no rescue), GST-tagged wild-type Musashi1 (Msi WT) or the non-phosphorylatable mutant Musashi (Msi S322A) and scored when 50% of Msi WT expressing oocytes reached GVBD. Error bars represent SEM from four independent experiments (p<0.01, Student's t-test). The Msi WT and S322A proteins were expressed to equivalent levels in the rescue assay as assessed by GST western blotting (lower panel). Western blot of the same lysates with tubulin antiserum confirmed equivalent protein loading.

Mutational mimicry of Musashi1 S322 phosphorylation accelerates oocyte maturation (FIGS. 3C and 3D). Oocytes were injected with Musashi antisense oligonucleotides as described in (A) and subsequently reinjected with water (no rescue), GST wild-type Musashi1 (Msi WT) or the phosphomimetic mutant Musashi (Msi S322E) and maturation was scored when 50% of Musashi S322E expressing oocytes reached GVBD. Error bars represent SEM from three independent experiments (p<0.05, Student's t-test). The Msi WT and S322E proteins were expressed to equivalent levels in the rescue assay as assessed by GST western blot (lower panel). Western blot of the same lysates with MAP kinase antiserum confirmed equivalent protein loading.

Mutational mimicry of Musashi1 S322 phosphorylation accelerates activation of the Mos mRNA (FIG. 3E). Total RNA was isolated from GST Msi WT or Msi S332E mutant expressing oocytes. Samples were prepared when 50% of Msi S322E oocytes reached GVBD and segregated based on whether they had or had not completed GVBD. Samples from time matched Msi WT and water injected (no rescue) were also prepared and endogenous Mos mRNA polyadenylation assessed.

Mutational mimicry of Musashi S322 phosphorylation enhances Mos protein accumulation (FIG. 3F). Protein lysates were isolated from GST Msi WT or Msi 5322E expressing oocytes and probed for Mos protein levels by western blot. Samples were prepared when 50% of the injected oocytes reached GVBD and segregated. Note the Msi 5322E expressing oocytes display higher Mos protein levels despite being harvested 30 minutes prior to Msi WT oocytes. These findings indicate that the progesterone-stimulated phosphorylation of S322 activates Musashi function, resulting in target mRNA translation and oocyte maturation.

### Example 5

### Musashi Function is Necessary for Ringo-Induced Early Class mRNA Translational Activation

The S322 residue of Musashi is located within a consensus motif for a proline-directed kinase, such as MAP kinase. However, Mos, the primary MAP kinase activator in oocytes, is itself regulated through translational activation by Musashi, suggesting that Musashi must be initially activated by a MAP kinase-independent mechanism. Consistently, treatment of oocytes with the MAP kinase signaling inhibitor UO126 does not prevent initial polyadenylation or translation of the Mos mRNA.

Alternate progesterone-stimulated, proline-directed kinases are the cyclin-dependent kinases (CDK1 and CDK2) that are initially activated through synthesis of the non-cyclin protein, Ringo. Ringo synthesis has been reported to precede and be necessary for translation of the Mos mRNA, positioning Ringo/CDK to act upstream of Musashi activation. Consistent with prior studies, we observed that ectopic expression of Ringo was sufficient to drive translational activation of the Mos mRNA independently of progesterone stimulation (FIG. 4A). Immature oocytes were injected with RNA encoding dominant negative Musashi (N-Msi) or water, incubated to allow expression of the N-Msi protein and subsequently re-injected with RNA encoding Ringo (FIG. 4A). Total RNA was prepared at various times after Ringo RNA injection and progression through maturation (GVBD) assessed. The time required for 50% of the oocyte population to reach GVBD was significantly delayed in N-Msi expressing oocytes (7 hours vs. 4 hours). Polyadenylation of the endogenous Mos mRNA was assessed. Ectopic expression of Ringo resulted in phosphorylation of Musashi on S322, prior to GVBD (FIG. 4B). Immature oocytes were injected with RNA encoding GST tagged Musashi1 and incubated overnight. The oocytes were then left untreated (Imm) or re-injected with RNA encoding Ringo or Ringo D83A as indicated and time matched protein lysates prepared when 50% of Ringo-injected oocytes completed GVBD. Ringo injected oocytes were segregated based on whether they had (+) or had not completed GVBD (−). Ringo D83A injected oocytes did not mature. Western blotting was performed with appropriate antisera to analyze phosphorylation of GST-Musashi1 S322, phosphorylation (activation) of MAP kinase, ectopic Ringo protein expression and expression of GST-Musashi1 as indicated.

Conversely, pre-treatment of oocytes with Ringo antisense oligonucleotides blocked progesterone-induced Musashi S322 phosphorylation (FIG. 4C). Immature oocytes were co-injected with RNA encoding GST-Musashi1 and either control antisense oligonucleotides (Con AS) or antisense oligonucleotides targeting the endogenous Ringo mRNA (Ringo AS). The injected oocytes were then left untreated (Imm) or stimulated with progesterone (+prog). Con AS oocytes were segregated when the 50% of the population reached GVBD (3.5 hours) along with time matched Ringo AS injected oocytes and protein lysates were analyzed for Musashi1 S322 phosphorylation and GST-Musashi expression by western blot. No maturation of Ringo AS injected oocytes was observed at the time points analyzed.

### Example 6

### Ringo/CDK and MAP Kinase Direct Musashi Phosphorylation on S322

MAP kinase signaling induces Musashi1 S322 phosphorylation independently of CDK (FIG. 5B). Immature oocytes were injected with RNA encoding GST tagged Musashi1 in the presence or absence of RNA encoding the CDK inhibitor Wee107 and incubated overnight. Oocytes were then split into pools and left untreated, injected with RNA encoding vRaf or stimulated with progesterone as indicated. Protein lysates were analyzed by western blot for Musashi1 phosphorylation on S322, MAP kinase phosphorylation (indicative of activation), Cdc2 phosphorylation (indicative of inactive cyclin B/CDK) and relative GST-Musashi1 expression.

To determine if Musashi played a role in MAP kinase-mediated Mos mRNA translation, Musashi S322 phosphorylation was examined in oocytes treated with the MAP kinase kinase (MEK) inhibitor, UO126. Inhibition of MAP kinase signaling resulted in significant attenuation of progesterone-stimulated Musashi S322 phosphorylation (FIG. 5B). Conversely, expression of an activator of MAP kinase signaling (vRaf) induced Musashi S322 phosphorylation in the absence of progesterone stimulation (FIG. 5A). Notably, the induction of Musashi S322 phosphorylation, through vRaf expression, was not inhibited in oocytes expressing Wee107, indicating that MAP kinase signaling is sufficient for Musashi S322 phosphorylation, independently of CDK activity (FIG. 5A). However, a low level of Musashi S322 phosphorylation was reproducibly observed in UO126 treated oocytes (FIG. 5B), supporting a role for both MAP kinase-independent, Ringo/CDK signaling and MAP kinase-dependent signaling in Musashi phosphorylation. Progesterone-stimulated Musashi1 S322 phosphorylation was mediated by both MAP kinase-dependent and MAP kinase-independent signaling (FIG. 5D). Immature oocytes were injected with RNA encoding GST-Musashi1 and incubated overnight, then treated with U0126 to inhibit MAP kinase signaling (+) or DMSO vehicle control (−) for 30 minutes prior to progesterone addition. Time matched protein lysates were prepared when 50% of the control oocytes reached GVBD. Control oocytes were segregated based on whether they had (+) or had not (−) completed GVBD. Lysates were analyzed by western blot for phosphorylation of Musashi1 on S322, phosphorylation (activation) of MAP kinase, total MAP kinase and expression of GST-Musashi1 in the same samples.

The results indicate that Ringo/CDK-dependent phosphorylation of Musashi S322 activates Musashi early in response to progesterone stimulation resulting in Mos synthesis and MAP kinase activation, which then further mediates phosphorylation and activation of additional Musashi protein via positive feedback (FIG. 5A).

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

What is claimed is:

1. An antibody which selectively binds to a polypeptide of the Musashi family.

2. The antibody of claim 1, wherein the polypeptide comprises an amino acid sequence having about 85% identity to a sequence selected from the group consisting of SEQ ID NO: 11, SEQ ID NO: 12, SEQ ID NO: 13, SEQ ID NO: 14, SEQ ID NO: 15, SEQ ID NO: 16, and SEQ ID NO: 17.

3. The antibody of claim 1, wherein the polypeptide has been post-translationally modified.

4. A composition comprising at least one Musashi indicator, wherein the post-translational status of Musashi protein is detected.

5. The compositions of claim 4, wherein the Musashi indicator is selected from the group consisting of antibody, oligonucleotide, protein, polypeptide, small molecule and combinations thereof.

6. A method of detecting post-translational status of Musashi protein comprising:
a. contacting a sample with at least one Musashi indicator; and,
b. detecting the post-translational status of Musashi protein.

7. The method of claim 6, wherein the Musashi indicator is selected from the group consisting of antibody, oligonucleotide, protein, polypeptide, small molecule and combinations thereof.

8. A method of modulating Musashi protein activity comprising contacting a sample with a Musashi agent that modulates Musashi activity.

9. The method of claim 8, wherein the Musashi agent is selected from the group consisting of antibody, oligonucleotide, protein, polypeptide, small molecule and combinations thereof.

10. A method of treating at least one symptom or sign of proliferative pathologies in a subject which comprises administering an effective amount of at least one Musashi agent to the patient.

11. The method of claim 10, wherein the Musashi agent is selected from the group consisting of antibody, oligonucleotide, protein, polypeptide, small molecule and combinations thereof.
